(19) United States
(12) Patent Application Publication
(10) Pub. No.: US 2019/0282141 A1
(43) Pub. Date: Sep. 19, 2019

(54) FLUID ANALYTE DETECTION SYSTEMS AND METHODS

(71) Applicant: OptiScan Biomedical Corporation, Hayward, CA (US)

(72) Inventors: James D. Causey, III, Simi Valley, CA (US); Michael Adams, Hayward, CA (US); Eugene Russiyanov, Hayward, CA (US); Brian Orr, Hayward, CA (US)

(21) Appl. No.: 16/351,425

(22) Filed: Mar. 12, 2019

Related U.S. Application Data
(60) Provisional application No. 62/642,561, filed on Mar. 13, 2018.

(51) Int. Cl. A61B 5/145; A61B 5/1455; A61B 5/1486

(52) U.S. Cl. CPC A61B 5/14532; A61B 5/14546; A61B 2562/0233; A61B 5/1486; A61B 5/1455

(57) ABSTRACT

Various embodiments disclosed herein relate to measuring multiple analytes in a medical system configured to draw in biological fluids. The system can include a fluid handling network configured to receive a fluid sample drawn from a patient and to deliver at least a portion of the fluid sample to an analyte measurement system. The measurement system can include a flow cell incorporated in line in a fluid system, shaped to allow a light source to directly abut the flow cell and cause radiation to pass through to a spectrometer on the other side.

### CROSS-REFERENCE TO RELATED APPLICATIONS

This application claims the benefit under 35 U.S.C. § 119(e) of U.S. Provisional Application No. 62/642,561 (Atty. Docket No. OPTIS.280PR), titled “FLUID ANALYTE DETECTION SYSTEMS AND METHODS,” filed on Mar. 13, 2018. The entire disclosure of each of the above-identified applications is incorporated by reference herein and made part of this specification.

### BACKGROUND

### Field

Some embodiments of the disclosure relate generally to methods and devices for determining a concentration of an analyte in a sample, such as an analyte in a sample of bodily fluid, as well as methods and devices which can be used to support the making of such determinations. Some embodiments of the disclosure relate to a sample cell for measurements performed on a sample fluid.

### Related Art

It can be advantageous to measure the levels of certain analytes, such as glucose or lactate, in a bodily fluid such as blood. This can be done, for example, in a hospital or clinical setting when there is a risk that the levels of certain analytes may move outside a desired range, which in turn can jeopardize the health of a patient. Currently known systems for analyte monitoring in a hospital or clinical setting may suffer from various drawbacks.

### BRIEF DESCRIPTION OF THE DRAWINGS

The following drawings and the associated descriptions are provided to illustrate embodiments of the present disclosure and do not limit the scope of the claims.

FIG. 1 is a block diagram of a system for sampling and analyzing fluid samples.

FIG. 2 schematically shows an example embodiment of a fluid analysis system that includes a disposable system and a main system.

FIG. 3 schematically shows an example fluid analysis system that may reflect many of the features described above with regard to the sampling and analysis system and/or the fluid analysis system above.

FIG. 4 shows an isometric view of an example fluid cell component that can be used to construct a flow cell.

FIG. 5 shows a top view of the fluid cell component shown in FIG. 4.

FIG. 6 shows a cross section of the embodiment of FIG. 5 along an axis.

FIG. 7 shows an isometric view of the fluid cell component from below.

FIG. 8 shows an isometric view of a solid lower fluid cell component 600 and a translucent wireframe upper fluid cell component 600.

FIG. 9 shows the embodiment of FIG. 8 where including a solid upper fluid cell component.

FIG. 10 shows a bottom solid view of the embodiment described in FIG. 8.

FIG. 11 shows a translucent bottom view of the embodiment of FIG. 8.

FIG. 12 shows an isometric view of a solid lower fluid cell component and a translucent upper fluid cell component.

FIG. 13 shows a cross section view of the assembled fluid cell shown in FIG. 8.

FIG. 14 shows the first end before the insertion of adhesive and/or tubing therein.

FIG. 15 shows the first end of the assembled fluid cell after insertion of a tube and adhesive.

FIG. 16 schematically illustrates the layout of an example embodiment of a fluid system.

FIG. 17 shows “Glucose” concentration of 150 mg/dL, while the graphic 2412 shows “Lactate” concentration of 0.5 mmol/L.

FIG. 18 illustrates the visual appearance of certain embodiments of the user interface.

FIG. 19 illustrates the visual appearance of certain embodiments of the user interface.

FIG. 20 shows an analyte detection system that is connected to remote stations over a network.

FIG. 21 shows an analyte detection system that is connected to remote stations over a network.

FIG. 22 illustrates an embodiment of a process for assuring calibration com illustrates an embodiment of a process for assuring calibration compliance within the analyte detection system by utilizing a lockout mechanism pliance within the analyte detection system by utilizing a lockout mechanism.

FIG. 23 illustrates one embodiment of a software update system.

FIG. 24 shows a process flow diagram of a preferred software update process.

FIG. 25 schematically illustrates the layout of an example embodiment of a fluid system.

FIG. 26 shows example graphs showing the first four principle components of difference spectra obtained according to an embodiment of the method.

FIG. 27 shows additional example graphs showing the first four principle components of difference spectra obtained according to an embodiment of the method.

These and other features will now be described with reference to the drawings summarized above. The drawings and the associated descriptions are provided to illustrate embodiments and not to limit the scope of any claim. Throughout the drawings, reference numbers may be reused to indicate correspondence between referenced elements. In addition, where applicable, the first one or two digits of a reference numeral for an element can frequently indicate the figure number in which the element first appears.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The systems and methods discussed herein can be used anywhere, including, for example, in laboratories, hospitals, healthcare facilities, intensive care units (ICUs), or residences. Moreover, the systems and methods discussed herein can be used for invasive techniques, as well as non-invasive techniques or techniques that do not involve a body or a patient such as, for example, in vitro techniques.

### Sampling and Analysis Systems

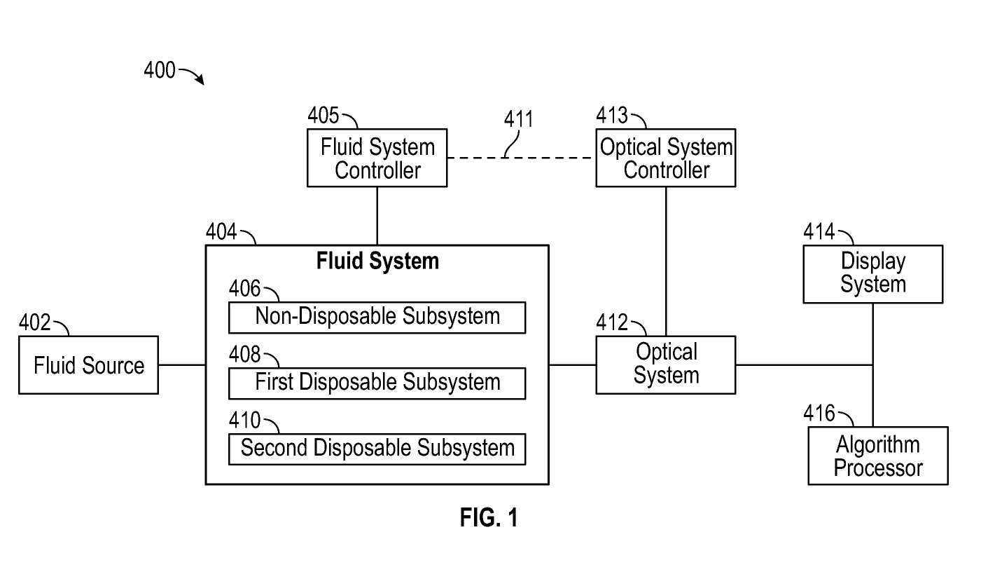

FIG. 1 is a block diagram of a system 400 for sampling and analyzing fluid samples. The monitoring device 102 can comprise such a system. The system 400 can include a fluid source 402 connected to a fluid-handling system 404. The fluid-handling system 404 includes fluid passageways and other components that direct fluid samples. Samples can be withdrawn from the fluid source 402 and analyzed by an optical system 412. The fluid-handling system 404 can be controlled by a fluid system controller 405, and the optical system 412 can be controlled by an optical system controller 413. The sampling and analysis system 400 can also include a display system 414 and an algorithm processor 416 that assist in fluid sample analysis and presentation of data.

In some embodiments, the sampling and analysis system 400 is a mobile point-of-care apparatus that monitors physiological parameters such as, for example, blood glucose concentration. Components within the system 400 that may contact fluid and/or a patient, such as tubes and connectors, can be coated with an antibacterial coating to reduce the risk of infection. Connectors between at least some components of the system 400 can include a self-sealing valve, such as a spring valve, in order to reduce the risk of contact between port openings and fluids, and to guard against fluid escaping from the system. Other components can also be included in a system for sampling and analyzing fluid in accordance with the described embodiments.

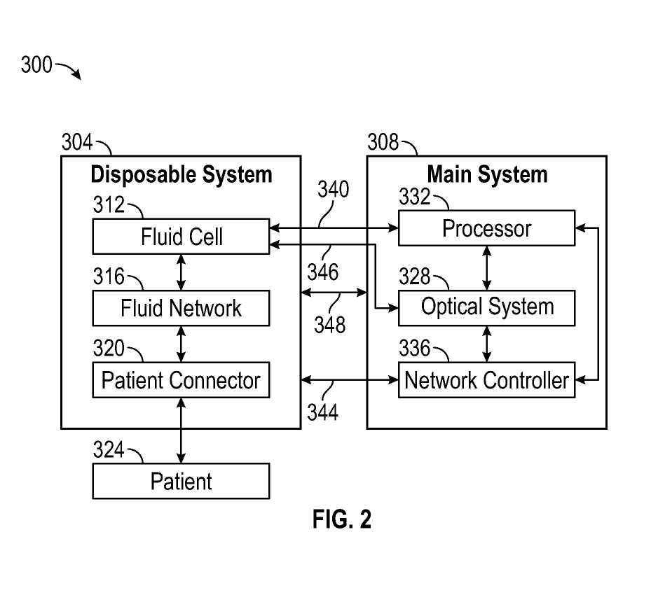

The sampling and analysis system 400 can include a fluid source 402 (or more than one fluid source) that contain(s) fluid to be sampled. The fluid-handling system 404 of the sampling and analysis system 400 is connected to, and can draw fluid from, the fluid source 402. The fluid source 402 can be, for example, a blood vessel such as a vein or an artery, a container such as a decanter, flask, beaker, tube, cartridge, test strip, etc., or any other corporeal or extracorporeal fluid source. For example, in some embodiments, the fluid source 402 may be a vein or artery in the patient 324 (see, e.g., FIG. 2). In other embodiments, the fluid source 402 may comprise an extracorporeal container 350 of fluid delivered to the system 400 for analysis. The fluid to be sampled can be, for example, blood, plasma, interstitial fluid, lymphatic fluid, or another fluid. In some embodiments, more than one fluid source can be present, and more than one fluid and/or type of fluid can be provided.

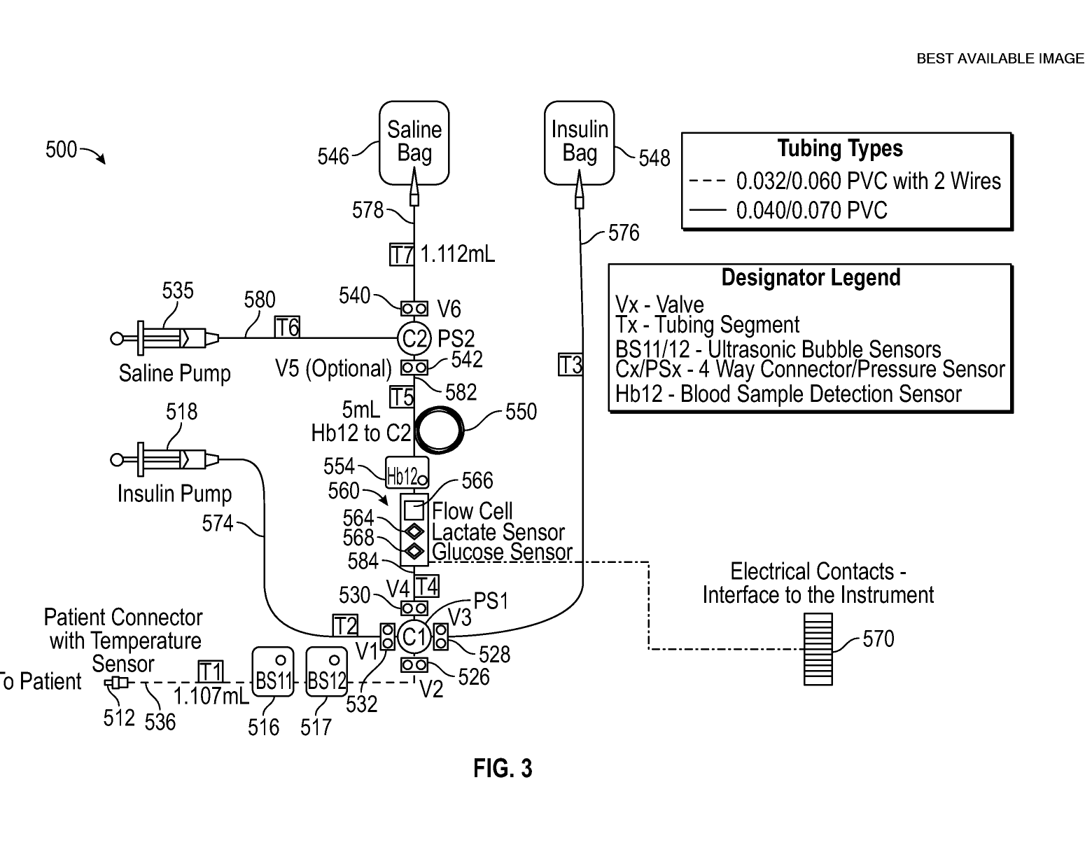

In some embodiments, the fluid-handling system 404 withdraws a sample of fluid from the fluid source 402 for analysis, passes it through a flow cell having multiple measurement modalities (e.g., optical and electrochemical) and passes it back to the patient without disturbing the structure of the fluid components (e.g., blood cells). In some embodiments, the fluid-handling system 404 withdraws a sample of fluid from the fluid source 402 for analysis, centrifuges (or otherwise separates, for example, by lysing) at least a portion of the sample, and prepares at least a portion of the sample for analysis by an optical sensor such as a spectrophotometer (which can be part of an optical system 412, for example). In certain embodiments, the fluid drawn from the fluid source 402 can be analyzed using optical interrogation techniques, enzymatic sensors (see, e.g., FIG. 3), and/or other interrogation methods. These functions can be controlled by a fluid system controller 405, which can also be integrated into the fluid-handling system 404. The fluid system controller 405 can also control the additional functions described below. In some embodiments, the sample can be withdrawn continuously or substantially continuously at certain time intervals with a given period. The time intervals at which the sample is withdrawn can be periodic or aperiodic and range from approximately 1 minute to approximately 15 minutes (e.g., the sample can be withdrawn at time intervals of 1 minute, 5 minutes, 10 minutes or 15 minutes). In some embodiments, the sample can be withdrawn at discrete time intervals (e.g., once every 30 minutes, once every 45 minutes or once every hour).

The duration of time over which the sample of fluid is withdrawn, referred to as “draw period”, may be set to avoid clinical drawbacks, and/or it can be varied according to a health-care provider's wishes. For example, in some embodiments, fluid may be continuously withdrawn into the sampling and analysis system 400 over a draw period lasting approximately 10 seconds to approximately 5 minutes.

In some embodiments, the amount of sample withdrawn from the fluid source 402 can be small. For example, in some embodiments, the volume of sample withdrawn from the fluid source can be between approximately 1.0 ml and approximately 10.0 ml in a draw period (e.g. 2.0 ml-6.0 ml of sample can be withdrawn in a draw period of approximately 1 minute). In some embodiments, the amount of sample withdrawn can be in the range of approximately 20 ml/day to approximately 500 ml/day. In some embodiments, the amount of sample withdrawn can be outside this range.

In some embodiments, at least a portion of the sample is returned to the fluid source 402. At least some of the sample, such as portions of the sample that are mixed with other materials or portions that are otherwise altered during the sampling and analysis process, or portions that, for any reason, are not to be returned to the fluid source 402, can also be placed in a waste bladder (not shown in FIG. 1). The waste bladder can be integrated into the fluid-handling system 404 or supplied by a user of the system 400. However, in some embodiments, all or substantially all of the sample is returned to the fluid source 402. The fluid-handling system 404 can also be connected to a saline source, a detergent source, and/or an anticoagulant source, each of which can be supplied by a user, attached to the fluid-handling system 404 as additional fluid sources, and/or integrated into the fluid-handling system 404.

Components of the fluid-handling system 404 can be modularized into one or more non-disposable, disposable, and/or replaceable subsystems. In the embodiment shown in FIG. 1, components of the fluid-handling system 404 are separated into a non-disposable subsystem 406, a first disposable subsystem 408, and a second disposable subsystem 410. In other embodiments not shown, the fluid-handling system 404 may include only the first disposable subsystem 408 and not the second disposable subsystem 410.

The non-disposable subsystem 406 can include components that, while they may be replaceable or adjustable, do not generally require regular replacement during the useful lifetime of the system 400. In some embodiments, the non-disposable subsystem 406 of the fluid-handling system 404 includes one or more reusable valves and sensors. For example, the non-disposable subsystem 406 can include one or more valves (or non-disposable portions thereof), (e.g., pinch-valves, rotary valves, etc.), sensors (e.g., ultrasonic bubble sensors, non-contact pressure sensors, optical blood dilution sensors, etc.). The non-disposable subsystem 406 can also include one or more pumps (or non-disposable portions thereof). For example, some embodiments can include pumps available from Hospira. In some embodiments, the components of the non-disposable subsystem 406 are not directly exposed to fluids and/or are not readily susceptible to contamination.

The first and/or second disposable subsystems 408, 410 can include components that are regularly replaced under certain circumstances in order to facilitate the operation of the system 400. For example, the first disposable subsystem 408 can be replaced after a certain period of use, such as a few days, has elapsed. Replacement may be necessary, for example, when a bladder within the first disposable subsystem 408 is filled to capacity. Such replacement may mitigate fluid system performance degradation associated with and/or contamination wear on system components.

In some embodiments, the first disposable subsystem 408 includes components that may contact fluids such as patient blood, saline, flushing solutions, anticoagulants, and/or detergent solutions. For example, the first disposable subsystem 408 can include one or more tubes, fittings, cleaner pouches and/or waste bladders. The components of the first disposable subsystem 408 can be sterilized in order to decrease the risk of infection and can be configured to be easily replaceable.

In some embodiments, the second disposable subsystem 410 can be designed to be replaced under certain circumstances. For example, in applicable embodiments, the second disposable subsystem 410 can be replaced when the patient being monitored by the system 400 is changed. The components of the second disposable subsystem 410 may not need replacement at the same intervals as the components of the first disposable subsystem 408. For example, the second disposable subsystem 410 can include a sample holder and/or at least some components of a centrifuge, components that may not become filled or quickly worn during operation of the system 400. Replacement of the second disposable subsystem 410 can decrease or eliminate the risk of transferring fluids from one patient to another during operation of the system 400, enhance the measurement performance of system 400, and/or reduce the risk of contamination or infection.

In some embodiments, the sample holder of the second disposable subsystem 410 receives the sample obtained from the fluid source 402 via fluid passageways of the first disposable subsystem 408. The sample holder is a container that can hold fluid for the centrifuge (and/or other sample conditioning element such as an ultrasonic lyser) and can include a window to the sample for analysis by a spectrometer. It can also or alternatively include access to the sample for enzymatic and/or electrochemical measurement. In some embodiments, the sample holder includes windows that are made of a material that is substantially transparent to electromagnetic radiation in the near and/or mid-infrared range of the spectrum. For example, the sample holder windows can be made of calcium fluoride. In some embodiments, a sample holder can include walls formed from injection moldable material such as plastic.

An injector can provide a fluid connection between the first disposable subsystem 408 and the sample holder of the second disposable subsystem 410. In some embodiments, the injector can be removed from the sample holder to allow for free spinning of the sample holder during centrifugation. In some advantageous embodiments, no injector is required because no centrifugation is necessary.

In some embodiments, blood (or other sample fluids) can be conditioned prior to or subsequent to measurement. For example, blood cells can be lysed, separated, etc. Some lysing can be performed chemically. Some lysing can be performed with an ultrasonic emitter. In some embodiments, the components of the sample are separated by centrifuging for a period of time before measurements are performed by the optical system 412. For example, a fluid sample (e.g., a blood sample) can be centrifuged at a relatively high speed. The sample can be spun at a certain number of revolutions per minute (RPM) for a given length of time to separate blood plasma for spectral analysis. In some embodiments, the fluid sample is spun at about 7200 RPM. In some embodiments, the sample is spun at about 5000 RPM. In some embodiments, the fluid sample is spun at about 4500 RPM. In some embodiments, the fluid sample is spun at more than one rate for successive time periods. The length of time can be approximately 5 minutes. In some embodiments, the length of time is approximately 2 minutes. Separation of a sample into the components (or other sample conditioning) can permit measurement of solute (e.g., glucose) concentration in plasma, for example, without interference from other blood components. Post-separation measurement can have advantages over a solute measurement taken from whole blood because the proportions of plasma to other components need not be known or estimated in order to infer plasma glucose concentration. In some embodiments, the separated plasma can be analyzed electrically using one or more electrodes instead of, or in addition to, being analyzed optically. This analysis may occur within the same device, or within a different device. For example, in certain embodiments, an optical analysis device can condition a sample (e.g., separate blood into components), analyze the sample, and then allow the sample and/or components thereof to be transported to another analysis device that can further analyze the sample and/or components (e.g., using electrical and/or electrochemical measurements).

Certain embodiments employ one or more enzymatic sensors to obtain measurements of one or more analytes within the fluid sample. In such embodiments a centrifuge and/or other means for plasma separation can be omitted. Fluid conditioning can include adding an anticoagulant, such as, for example, heparin, to the sample to prevent clotting. The fluid-handling system 404 can be used with a variety of anticoagulants, including anticoagulants supplied by a hospital or other user of the monitoring system 400. In some embodiments, no anticoagulant is necessary and is avoided such that blood can be returned to a patient after measurement. A detergent solution formed by mixing detergent powder from a pouch connected to the fluid-handling system 404 with saline can be used to periodically clean residual protein and other sample remnants from one or more components of the fluid-handling system 404, such as the sample holder. Sample fluid to which anticoagulant has been added and used detergent solution can be transferred into the waste bladder.

The system 400 shown in FIG. 1 includes an optical system 412 that can measure optical properties (e.g., transmission) of a fluid sample (or a portion thereof). In some embodiments, the optical system 412 measures transmission in the near and/or mid-infrared range of the spectrum. In some embodiments, the optical system 412 includes a spectrometer that measures the transmission of broadband infrared light through a portion of a sample holder filled with fluid. The spectrometer need not come into direct contact with the sample. As used herein, the term “sample holder” is a broad term that carries its ordinary meaning as an object that can provide a place for fluid. The fluid can enter the sample holder by flowing.

In some embodiments, an optical system 412 includes a filter wheel that contains one or more filters. In some embodiments, more than ten filters can be included, for example twelve or fifteen filters. In some embodiments, more than 20 filters (e.g., twenty-five filters) are mounted on the filter wheel. The optical system 412 includes a light source that passes light through a filter and the sample holder to a detector. In some embodiments, a stepper motor moves the filter wheel in order to position a selected filter in the path of the light. An optical encoder can also be used to finely position one or more filters. In some embodiments, one or more tunable filters may be used to filter light into multiple wavelengths. The one or more tunable filters may provide the multiple wavelengths of light at the same time or at different times (e.g., sequentially). In some advantageous embodiments, no filters are needed because none, one, or very few wavelengths are required for analysis.

The light source included in the optical system 412 may emit radiation in the ultraviolet, visible, near-infrared, mid-infrared, and/or far-infrared regions of the electromagnetic spectrum. In some embodiments, the light source can be a broadband source that emits radiation in a broad spectral region (e.g., from about 1500 nm to about 6000 nm). In other embodiments, the light source may emit radiation at certain specific wavelengths. The light source may comprise one or more light emitting diodes (LEDs) emitting radiation at one or more wavelengths in the radiation regions described herein. In other embodiments, the light source may comprise one or more laser modules emitting radiation at one or more wavelengths. The laser modules may comprise a solid state laser (e.g., a Nd:YAG laser), a semiconductor based laser (e.g., a GaAs and/or InGaAsP laser), and/or a gas laser (e.g., an Ar-ion laser). In some embodiments, the laser modules may comprise a fiber laser. The laser modules may emit radiation at certain fixed wavelengths. In some embodiments, the emission wavelength of the laser module(s) may be tunable over a wide spectral range (e.g., about 30 nm to about 100 nm). In some embodiments, the light source included in the optical system 412 may be a thermal infrared emitter. The light source can comprise a resistive heating element, which, in some embodiments, may be integrated on a thin dielectric membrane on a micromachined silicon structure. In one embodiment the light source is generally similar to the electrical modulated thermal infrared radiation source, IRSource™, available from the Axetris Microsystems division of Leister Technologies, LLC (Itasca, Ill.).

The optical system 412 can be controlled by an optical system controller 413. The optical system controller can, in some embodiments, be integrated into the optical system 412. In some embodiments, the fluid system controller 405 and the optical system controller 413 can communicate with each other as indicated by the line 411. In some embodiments, the function of these two controllers can be integrated and a single controller can control both the fluid-handling system 404 and the optical system 412. Such an integrated control can be advantageous because the two systems are preferably integrated, and the optical system 412 is preferably configured to analyze the very same fluid handled by the fluid-handling system 404. Indeed, portions of the fluid-handling system 404 (e.g., the sample holder described above with respect to the second disposable subsystem 410 and/or at least some components of a centrifuge) can also be components of the optical system 412. Accordingly, the fluid-handling system 404 can be controlled to obtain a fluid sample for analysis by optical system 412, when the fluid sample arrives, the optical system 412 can be controlled to analyze the sample, and when the analysis is complete (or before), the fluid-handling system 404 can be controlled to return some or all of the sample to the fluid source 402 and/or discard some of the sample, as appropriate.

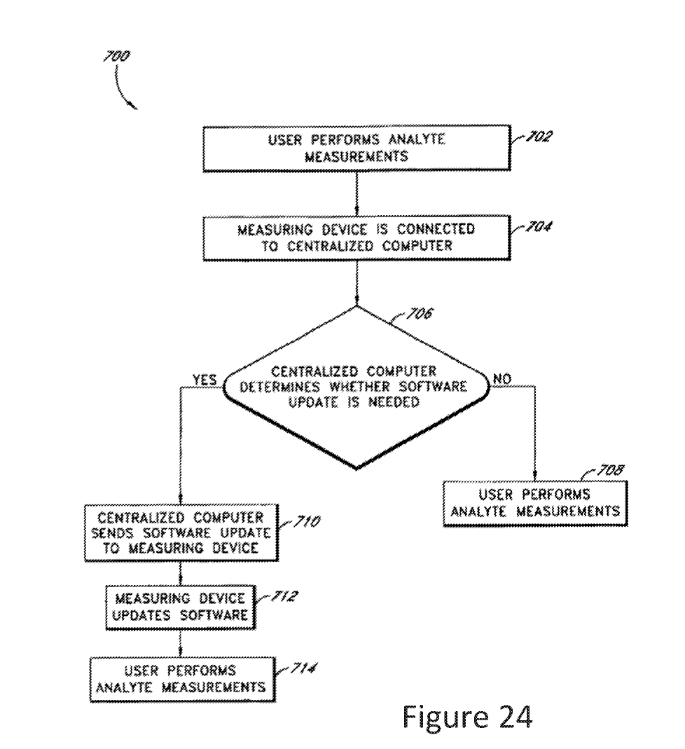

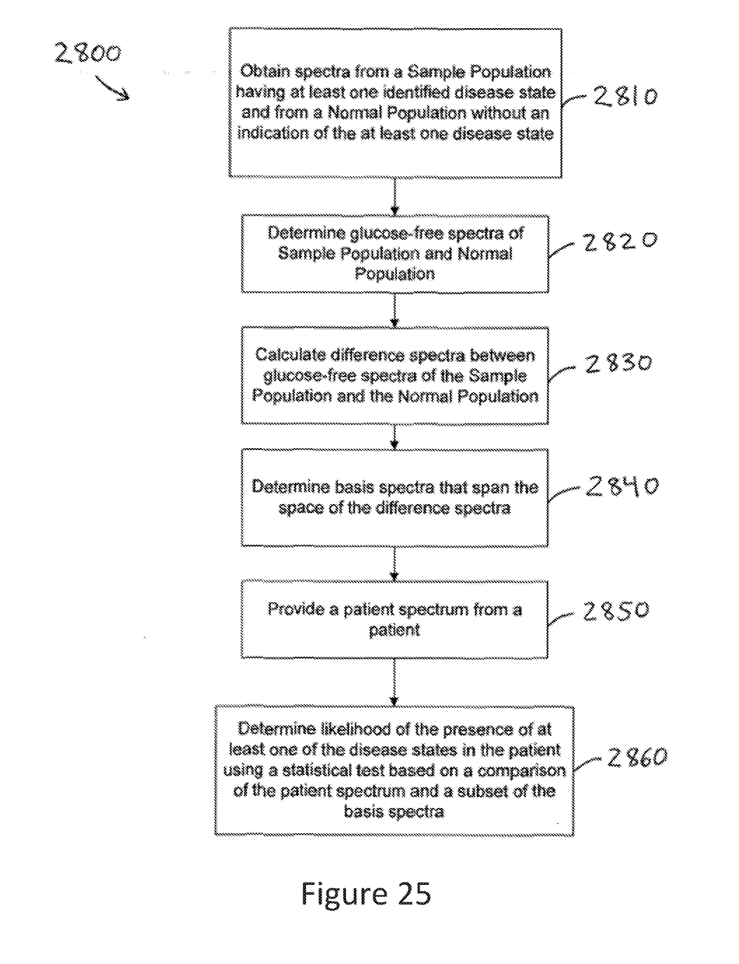

The system 400 shown in FIG. 1 includes a display system 414 that provides for communication of information to a user of the system 400. In some embodiments, the display 414 can be replaced by or supplemented with other communication devices that communicate in non-visual ways. The display system 414 can include a display processor that controls or produces an interface to communicate information to the user. The display system 414 can include a display screen. One or more parameters such as, for example, blood glucose concentration, system 400 operating parameters, and/or other operating parameters can be displayed on a monitor (not shown) associated with the system 400. Examples of how such information can be displayed is shown in FIGS. 24 and 25 of U.S. Patent Publication No. 2015/0045641, which is hereby incorporated by reference herein in its entirety for all purposes. In some embodiments, the display system 414 can communicate measured physiological parameters and/or operating parameters to a computer system over a communications connection.

The system 400 shown in FIG. 1 includes an algorithm processor 416 that can receive spectral information, such as optical density (OD) values (or other analog or digital optical data) from the optical system 412 and or the optical system controller 413. In some embodiments, the algorithm processor 416 calculates one or more physiological parameters and can analyze the spectral information. Thus, for example and without limitation, a model can be used that determines, based on the spectral information, physiological parameters of fluid from the fluid source 402. The algorithm processor 416, a controller that may be part of the display system 414, and any embedded controllers within the system 400 can be connected to one another with a communications bus.

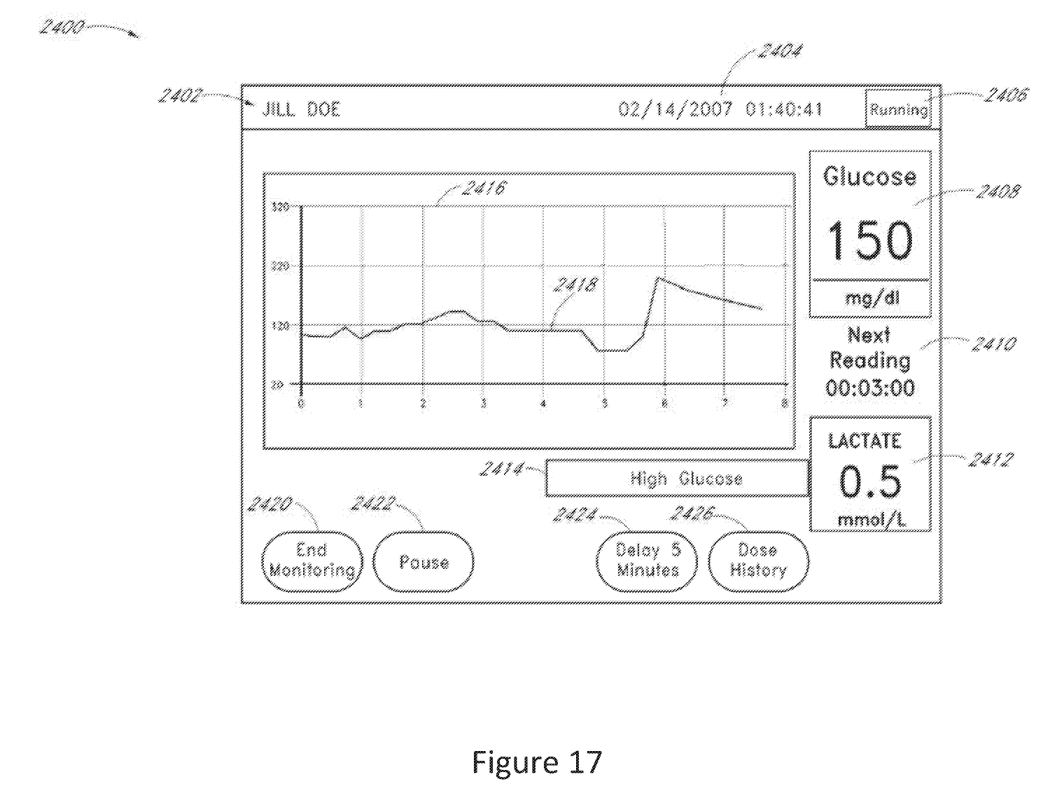

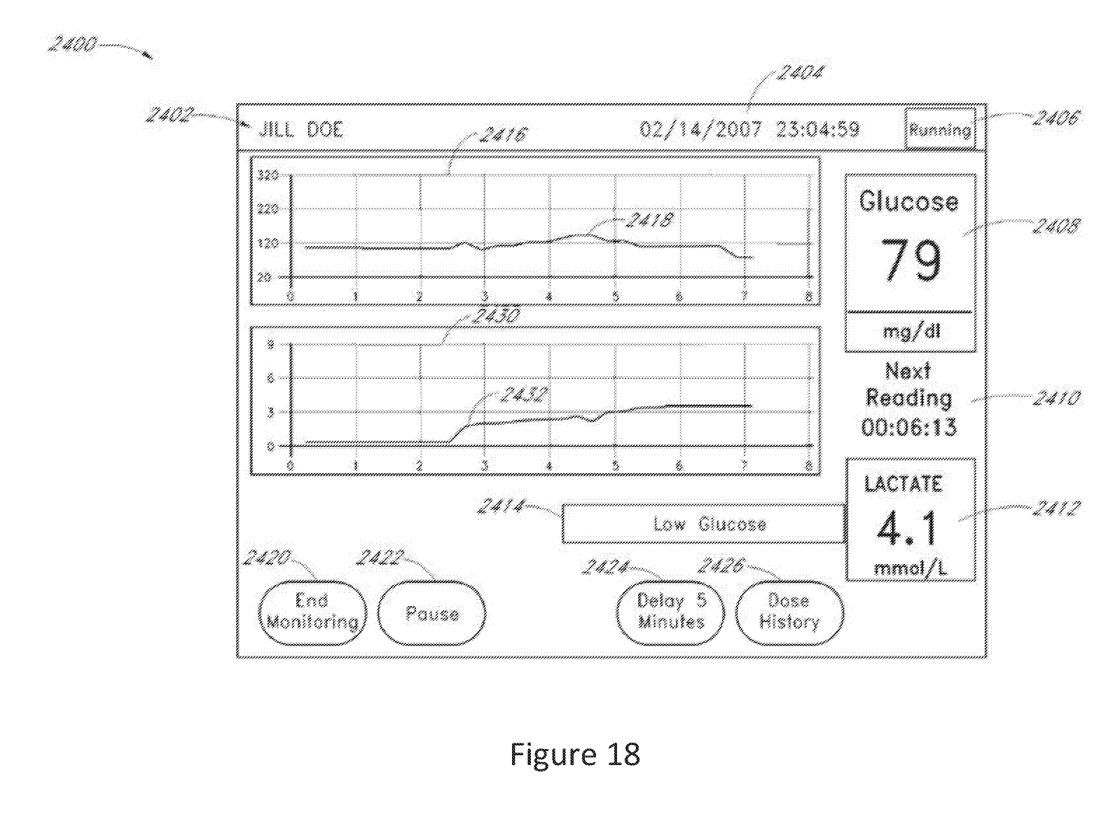

FIGS. 17 and 18 schematically illustrate the visual appearance of embodiments of the user interface 2400. The user interface 2400 may show patient identification information 2402, which can include patient name and/or a patient ID number. The user interface 2400 also can include the current date and time 2404. An operating graphic 2406 shows the operating status of the system 400. For example, as shown in FIGS. 24 and 25, the operating status is “Running,” which indicates that the system 400 is fluidly connected to the patient (“Jill Doe”) and performing normal system functions such as infusing fluid and/or drawing blood. The user interface 2400 can include one or more analyte concentration graphics 2408, 2412, which may show the name of the analyte and its last measured concentration. For example, the graphic 2408 in FIG. 17 shows “Glucose” concentration of 150 mg/dL, while the graphic 2412 shows “Lactate” concentration of 0.5 mmol/L. The particular analytes displayed and their measurement units (e.g., mg/dL, mmol/L, or other suitable unit) may be selected by the user. The size of the graphics 2408, 2412 may be selected to be easily readable out to a distance such as, e.g., 30 feet. The user interface 2400 may also include a next-reading graphic 2410 that indicates the time until the next analyte measurement is to be taken. In FIG. 17, the time until next reading is 3 minutes, whereas in FIG. 18, the time is 6 minutes, 13 seconds.

The user interface 2400 can include an analyte concentration status graphic 2414 that indicates status of the patient's current analyte concentration compared with a reference standard. For example, the analyte may be glucose and/or lactate, and the reference standard may be a hospital ICU's tight glycemic control (TGC). In FIG. 17, the status graphic 2414 displays “High Glucose” (and/or, in some embodiments, a similar reading for other analytes like lactate, such as “High Lactate”) because the glucose concentration (150 mg/dL) exceeds the maximum value of the reference standard. In FIG. 18, the status graphic 2414 displays “Low Glucose” (and/or, in some embodiments, a similar reading for other analytes like lactate, such as “Low Lactate”) because the current glucose concentration (79 mg/dL) is below the minimum reference standard. If the analyte concentration is within bounds of the reference standard, the status graphic 2414 may indicate normal (e.g., “Normal Glucose”), or it may not be displayed at all. The status graphic 2414 may have a background color (e.g., red, yellow, green, blue or some other color) when the analyte concentration exceeds the acceptable bounds of the reference standard.

In various implementations of the user interface 2400 a characteristic of the status graphic 2408 or 2412 displaying the concentration of the analyte can be varied (or otherwise emphasized) if the concentration of the analyte is predicted to be outside the acceptable bounds of the reference standard at a future time (e.g., less than or equal to about 5 minutes, less than or equal to about 10 minutes, less than or equal to about 15 minutes, less than or equal to about 20 minutes, less than or equal to about 30 minutes, less than or equal to about 45 minutes, less than or equal to about 60 minutes, less than or equal to about 90 minutes, less than or equal to about 120 minutes, etc.). The characteristic of the status graphic 2408 or 2412 can be a foreground and/or a background color. The characteristic of the status graphic 2408 or 2412 can be a visual state.

Various visual, auditory or other sensory approaches can be used to call attention to the display or a portion of the display. It can be advantageous to reduce anxiety of a patient while at the same time increasing awareness by a health-care provider. Such targeted but discrete emphasis can be accomplished, for example, by using visual cues that a health-care provider understands, but that do not add auditory alarms. Nevertheless, auditory alarms can be used for some particularly urgent circumstances. Visual emphasis effects can include foreground, background, or text colors, bright hues, increased color saturation or intensity, high contrast with surrounding visual colors, movement, animation, or dynamic displays, etc. Colors used can take advantage of existing or standardized cultural and/or medical meanings. For example, yellow can be a highly visual color that signifies caution but not an emergency. Red can signal something undesirable (or something that should be stopped), while green can signal something that is not dangerous or that is proceeding normally. These same colors can have other meanings in this or other contexts. Relative size or positioning of visual information can also be used for emphasis or de-emphasis. Larger symbols can call attention to more urgent or relevant information. In some embodiments, some colors (e.g., red and green) can be used for information about a current analyte value, while another color (e.g., yellow) can be used for information about a predicted or projected analyte value. Color, intensity, emphasis, etc. can change depending on the calculated certainty or uncertainty, and/or relative immediacy of a prediction, etc. These principles can apply to trend lines and other styles of data display (including tabular and bar graph displays, status graphics 2408 and 2412, etc. Three dimensional displays (with shading, contoured surfaces, multiple axes, movement into the screen to indicate forward movement in time, etc.) can be used and navigated using the user interface when multiple variables are being depicted.

For example, the status graphic 2408 or 2412 can have different foreground and/or background colors if the concentration of the analyte is predicted to be outside the acceptable bounds of the reference standard at a future time. For example, the status graphic 2408 or 2412 can have a yellow or an orange background color if the concentration of the analyte is predicted to be outside the acceptable bounds of the reference standard at a future time. The future time can be displayed, it can be adjustable, it can be modified through the display, and/or it can be set at a standard future time like 10 minutes, 30 minutes, 1 hour, 2 hours, etc. As another example, the status graphic 2408 or 2412 can have a green background or foreground color if the concentration of the analyte at the current measurement time and the predicted value for the concentration of the analyte at the future time is within the acceptable bounds of the reference standard. As yet another example, the status graphic 2408 or 2412 can be configured to blink and/or flash if the concentration of the analyte is predicted to be outside the acceptable bounds of the reference standard at a future time. Thus, for example, a nurse can be efficiently notified to change an insulin delivery rate based on measure glucose value and calculated or predicted rate of change.

For instance, the concentration of the analyte measured at the current measurement time may be within the acceptable bounds of the reference standard. However, if the value of the concentration of the analyte predicted to be outside the acceptable bounds of the reference standard at a future time (e.g., less than or equal to about 5 minutes, less than or equal to about 10 minutes, less than or equal to about 15 minutes, less than or equal to about 20 minutes, less than or equal to about 30 minutes, less than or equal to about 45 minutes, less than or equal to about 60 minutes, less than or equal to about 90 minutes, less than or equal to about 120 minutes, etc.), then the status graphic 2408 or 2412 can be emphasized according to the principles described herein (e.g., have a yellow or an orange background color and/or be configured to blink and/or flash, etc.) to indicate to the health care provider that the concentration of the analyte may be dangerous in the future, or outside an acceptable range of a reference standard. Accordingly, a health care provider (e.g., a doctor, a nurse or a care giver) can be alerted to change the dose of an infusion substance (e.g., insulin or dextrose) based on a dosing protocol to avoid concentration of the analyte from being outside the acceptable bounds of the reference standard at the future time. In various implementations, a patient monitoring system (e.g., system 400 of FIG. 1) can be configured to provide a suggested dose for the infusion substance based on the predicted value of the concentration of the analyte such as described in U.S. patent application Ser. No. 12/249,831 (Atty. Docket No. OPTIS.203A) and Ser. No. 12/559,328 (Atty. Docket No. OPTIS.247A), the entire disclosures of which are incorporated herein by reference, for all purposes.

The predicted value of the concentration of the analyte at a future time can be calculated using instructions stored in a processor (e.g., algorithm processor 416 of system 400 or the computer system 2646 of the system 2630) of a patient monitoring system (e.g., system 400 of FIG. 1).

The predicted value of the concentration of the analyte at a future time can be determined from one or more previous measurements of the analyte concentration. For example, various systems and methods of determining the predicted value of the concentration of the analyte at a future time as described in U.S. patent application Ser. No. 12/249,831 (Atty. Docket No. OPTIS.203A) and Ser. No. 12/559,328 (Atty. Docket No. OPTIS.247A)—the entire disclosures of which are incorporated herein by reference, for all purposes—can be employed to determine predicted value of the concentration of the analyte at a future time.

As another example, the predicted value of the concentration of the analyte at a future time can be determined from a rate of change in the concentration of the analyte calculated based on one or more previous measurements of the analyte concentration. For instance, the average rate of change in the concentration of the analyte over a past certain period of time (e.g., about 10 minutes, about 20 minutes, about 30 minutes, about 45 minutes, about 1 hour, etc.) can be determined and the value of the concentration of the analyte in the next certain period of time (e.g., about 10 minutes, about 20 minutes, about 30 minutes, about 45 minutes, about 1 hour, etc.) can be calculated assuming a predicted rate of change of concentration that is proportional to the determined average rate of change in the concentration of the analyte. In various implementations, the predicted rate of change of concentration can depend on the average rate of change in the concentration of the analyte over a past certain period of time and a dose of the infusion substance (e.g., insulin or dextrose) in that period of time.

In various implementations, the predicted rate of change of concentration can be depend on a first average rate of change in the concentration of the analyte over a first time interval previous to the current measurement time, a second average rate of change in the concentration of the analyte over a second time interval previous to the current measurement time, a third average rate of change in the concentration of the analyte over a third time interval previous to the current measurement time and so on. Additionally, the predicted rate of change of concentration can be depend on a first dose of the infusion substance (e.g., insulin or dextrose) in the first time interval, a second dose of the infusion substance in the second time interval, a third dose of the infusion substance in the second time interval and so on.

As another example, the predicted value of the concentration of the analyte at a future time can be determined from a trend in the values of the concentration of the analyte obtained in one or more previous measurements of the analyte concentration. For instance, the predicted value of the concentration of the analyte at a future time can be determined from a slope of the curve of at least two values of the concentration of the analyte obtained at a time previous to the current measurement time.

In various implementations, the patient monitoring system can be configured to determine the concentration of the analyte at time t1 and rate of change of concentration of the analyte from a previous measurement time t0 and calculate the predicted value of the concentration at a future time tn. The patient monitoring system can be configured to subsequently determine the concentration of the analyte at time t2 and rate of change of concentration of the analyte from the previous measurement time t1 and refine the predicted value of the concentration at the future time tn. In this manner the predicted value of the concentration at the future time tn can be calculated and refined on a moving window basis based on the rate of change in the concentration of the analyte at the current measurement from a previous measurement time. Such a moving window can advantageously “move” in sync with the advance of actual time by having an initial window opening and final window closing time, each of which is prescribed to be a fixed amount of time into the future. The window's parameters (e.g., the width of the window, its distance into the future from the present time, statistical error associated with the data it displays, etc.) can be adjusted by a user, or they can be set (e.g., by a manufacturer) to avoid confusion.

Various embodiments and aspects of this disclosure can be combined, in particular those referring to a graphic user interface. Accordingly, the disclosure herein relating to visual emphasis of predicted future values can be combined with the disclosure relating to adding annotations to a computer memory using a touch-screen with bookmarks and visual flags indicating when a note has been entered by a user. Thus, a flashing yellow indicator can be annotated (and the time stamp of the annotation, the identity of the source of the note, etc. can be recorded) when a doctor or nurse wishes to record that the alert was noticed but ignored or overridden because of a factor that does not play a part in the automatic machine algorithm that led to the alert. Other synergies existing between various aspects of the disclosure herein relating to user interfaces, displays and interactions therewith, and the ability of a system to provide accurate analyte information and projections.

The user interface 2400 can include one or more trend indicators 2416 that provide a graphic indicating the time history of the concentration of an analyte of interest. In FIGS. 17 and 18, the trend indicator 2416 comprises a graph of the glucose concentration (in mg/dL) versus elapsed time (in hours) since the measurements started. The graph includes a trend line 2418 indicating the time-dependent glucose concentration. In other embodiments, the trend line 2418 can include measurement error bars and may be displayed as a series of individual data points. In FIG. 18, the glucose trend indicator 2416 is shown as well as a trend indicator 2430 and trend line 2432 for the lactate concentration. In some embodiments, a user may select whether none, one, or both trend indicators 2416, 2418 are displayed. In some embodiments, one or both of the trend indicators 2416, 2418 may appear only when the corresponding analyte is in a range of interest such as, for example, above or below the bounds of a reference standard. A graphic user interface can also indicate predicted values using a trend display. For example, a dotted line can be displayed that projects into a portion of the graph depicting a future time. A separate value can be displayed for a given future time (although in many cases, this value may be less prominently displayed than the actual measured value). The trend line (or a portion thereof) can be displayed in various colors to indicate different levels of predicted future values. For example, a tip of the trend line can be displayed as yellow if it is predicted to fall within a dangerous range within the next hour.

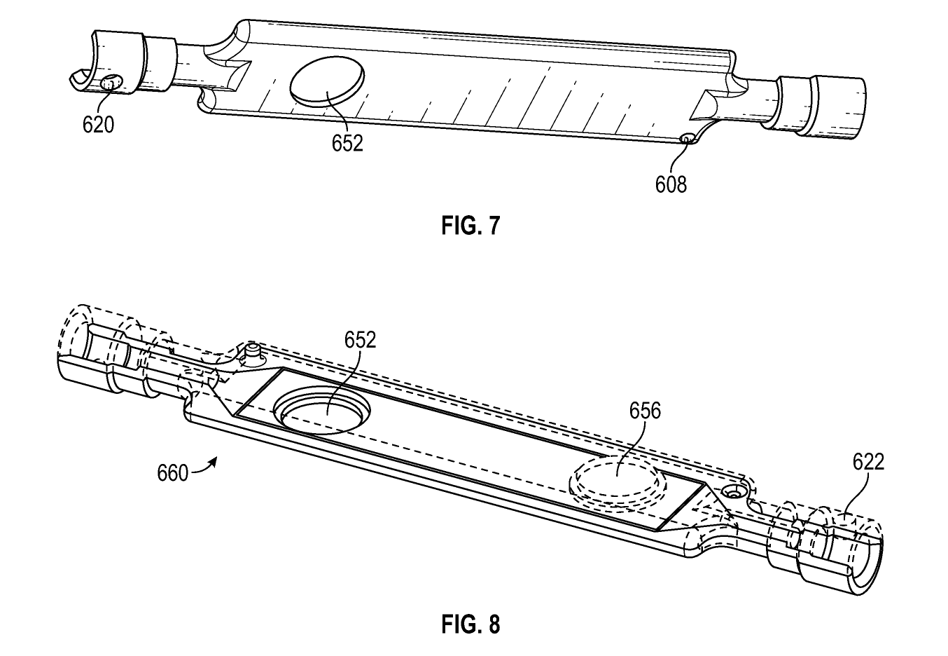

The user interface 2400 can include one or more buttons 2420-2426 that can be actuated by a user to provide additional functionality or to bring up suitable context-sensitive menus and/or screens. For example, in the embodiments shown in FIG. 17 and FIG. 18, four buttons 2420-2426 are shown, although fewer or more buttons are used in other embodiments. The button 2420 (“End Monitoring”) may be pressed when one or more removable portions (see, e.g., 710 of FIG. 7) are to be removed. In many embodiments, because the removable portions 710, 712 are not reusable, a confirmation window appears when the button 2420 is pressed. If the user is certain that monitoring should stop, the user can confirm this by actuating an affirmative button in the confirmation window. If the button 2420 were pushed by mistake, the user can select a negative button in the confirmation window. If “End Monitoring” is confirmed, the system 400 performs appropriate actions to cease fluid infusion and blood draw and to permit ejection of a removable portion (e.g., the removable portion 710).

The button 2422 (“Pause”) may be actuated by the user if patient monitoring is to be interrupted but is not intended to end. For example, the “Pause” button 2422 may be actuated if the patient is to be temporarily disconnected from the system 400 (e.g., by disconnecting the tubes 306). After the patient is reconnected, the button 2422 may be pressed again to resume monitoring. In some embodiments, after the “Pause” button 2422 has been pressed, the button 2422 displays “Resume.”

The button 2424 (“Delay 5 Minutes”) causes the system 400 to delay the next measurement by a delay time period (e.g., 5 minutes in the depicted embodiments). Actuating the delay button 2424 may be advantageous if taking a reading would be temporarily inconvenient, for example, because a health care professional is attending to other needs of the patient. The delay button 2424 may be pressed repeatedly to provide longer delays. In some embodiments, pressing the delay button 2424 is ineffective if the accumulated delay exceeds a maximum threshold. The next-reading graphic 2410 automatically increases the displayed time until the next reading for every actuation of the delay button 2424 (up to the maximum delay).

The button 2426 (“Dose History”) may be actuated to bring up a dosing history window that displays patient dosing history for an analyte or medicament of interest. For example, in some embodiments, the dosing history window displays insulin dosing history of the patient and/or appropriate hospital dosing protocols. A nurse attending the patient can actuate the dosing history button 2426 to determine the time when the patient last received an insulin dose, the last dosage amount, and/or the time and amount of the next dosage. The system 400 may receive the patient dosing history via wired or wireless communications from a hospital information system.

In other embodiments, the user interface 2400 can include additional and/or different buttons, menus, screens, graphics, etc. that are used to implement additional and/or different functionalities.

Some embodiments of the systems described herein (e.g., the system 400), as well as some embodiments of each method described herein, can include a computer program accessible to and/or executable by a processing system, e.g., a one or more processors and memories that are part of an embedded system. Indeed, the controllers may comprise one or more computers and/or may use software. Thus, as will be appreciated by those skilled in the art, various embodiments may be embodied as a method, an apparatus such as a special purpose apparatus, an apparatus such as a data processing system, or a carrier medium, e.g., a computer program product. The carrier medium carries one or more computer readable code segments for controlling a processing system to implement a method. Accordingly, various embodiments may take the form of a method, an entirely hardware embodiment, an entirely software embodiment or an embodiment combining software and hardware aspects. Furthermore, any one or more of the disclosed methods (including but not limited to the disclosed methods of measurement analysis, interferent determination, and/or calibration constant generation) may be stored as one or more computer readable code segments or data compilations on a carrier medium. Any suitable computer readable carrier medium may be used including a magnetic storage device such as a diskette or a hard disk; a memory cartridge, module, card or chip (either alone or installed within a larger device); or an optical storage device such as a CD or DVD.

FIG. 2 schematically shows an example embodiment of a fluid analysis system 300 that includes a disposable system 304 and a main system 308. The disposable system 304 can include a fluid cell 312, a fluid network 316, and/or a patient connector 320. The main system 308 can include a processor 332, an optical system 328, and/or a network controller 336. Other elements, such as those described above with reference to FIG. 1, can be included additionally or alternatively.

The fluid cell 312 can be in communication with a fluid network 316. The fluid network 316 can include one or more tubes, fittings, pumps, fluid interfaces, valves, sensors, connectors, cleaner pouches, and/or waste bladders. The components of the disposable system 304 may share one or more features with the first disposable subsystem 408 described above with reference to FIG. 1. The fluid network 316 can be in fluid communication with the patient connector 320. The patient connector 320 may be configured to interface with a catheter that is inserted in a body of a patient 324.

The fluid cell 312 can include one or more sensors and/or optical cells. The one or more sensors may include one or more enzymatic sensors. For example, the enzymatic sensors may include glucose and/or lactate sensors. Glucose and lactate can serve as indicators of health within a patient 324. For example, the presence of lactate above a certain threshold may indicate that a patient 324 requires urgent assistance. The fluid cell 312 can be in electrical communication with a processor 332 via a processor interface 340. The processor interface 340 may be configured to receive electrical and/or electro-chemical signals from the one or more sensors (e.g., enzymatic sensors) in the fluid cell 312.

The fluid cell 312 may also be in optical communication with the optical system 328 via an optical interface 346. The optical interface 346 may include a transparent or translucent boundary between the fluid sample and a light source. The boundary may include a portion (e.g., a body) of the fluid cell 312. For example, the fluid cell 312 may comprise a transparent material configured to allow the transmission of light therethrough. In this way, the fluid cell 312 can allow the optical system 328 to optically interrogate the fluid sample. This can serve advantageously as a non-invasive approach to determining an amount of an interferent/analyte (e.g., hemoglobin) within the fluid sample. The optical system 328 may share one or more features of the optical system 412 described above with reference to FIG. 1. The optical system 328 can have a light source, a light detector, and an optical path therebetween. The sample can be positioned in the optical path so that light from the light source (e.g., broadband light source) is transmitted through the sample to the light detector (e.g., a full-spectrum spectrometer), for measuring one or more analyte in the fluid sample. The optical system can include features that are the same as, or similar to, the optical system embodiments described herein. For example, the optical system 328 can have a reference detector, which can be similar to the reference detector 2036 described herein. The reference detector can be on the same side of the flow cell as the light source. A portion of the light from the light source can be sent to the reference detector, and a portion of the light from the light source can be sent through the flow cell to the measurement detector.

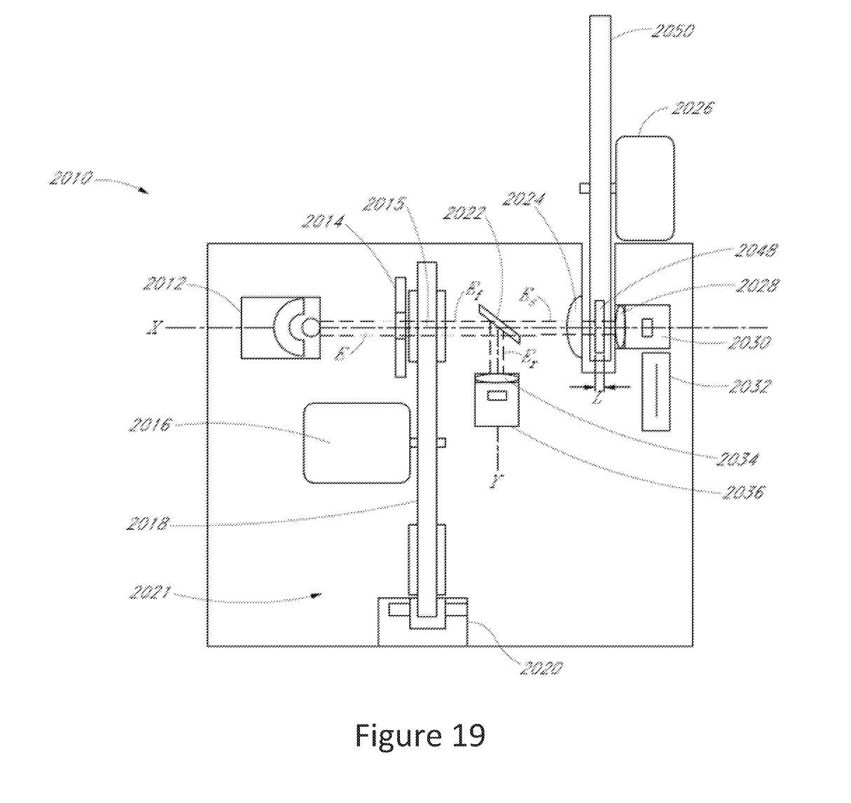

For example, with reference to FIG. 19, the filtered energy beam E_{f} propagates to a beamsplitter 2022 disposed along the optical axis X. The beamsplitter 2022 separates the filtered energy beam E_{f} into a sample beam Eₛ and a reference beam Eᵣ. The reference beam Eᵣ propagates along a minor optical axis Y, which in this embodiment is substantially orthogonal to the optical axis X. The energies in the sample beam Eₛ and the reference beam Eᵣ may comprise any suitable fraction of the energy in the filtered beam E_{f}. For example, in some embodiments, the sample beam Eₛ comprises about 80%, and the reference beam Eᵣ comprises about 20%, of the filtered beam energy E_{f}. A reference detector 2036 is positioned along the minor optical axis Y. An optical element 2034, such as a lens, may be used to focus or collimate the reference beam Eᵣ onto the reference detector 2036. The reference detector 2036 provides a reference signal, which can be used to monitor fluctuations in the intensity of the energy beam E emitted by the source 2012. Such fluctuations may be due to drift effects, aging, wear, or other imperfections in the source 2012. The algorithm processor 416 may utilize the reference signal to identify changes in properties of the sample beam Eₛ that are attributable to changes in the emission from the source 2012 and not to the properties of the fluid sample. By so doing, the analyzer 2010 may advantageously reduce possible sources of error in the calculated properties of the fluid sample (e.g., concentration). In other embodiments of the analyzer 2010, the beamsplitter 2022 is not used, and substantially all of the filtered energy beam E_{f} propagates to the fluid sample.

As illustrated in FIG. 19, the sample beam Eₛ propagates along the optical axis X, and a relay lens 2024 transmits the sample beam Eₛ into a sample cell 2048 so that at least a fraction of the sample beam Eₛ is transmitted through at least a portion of the fluid sample in the sample cell 2048. A sample detector 2030 is positioned along the optical axis X to measure the sample beam Es that has passed through the portion of the fluid sample. An optical element 2028, such as a lens, may be used to focus or collimate the sample beam Es onto the sample detector 2030. The sample detector 2030 provides a sample signal that can be used by the algorithm processor 416 as part of the sample analysis.

In the embodiment of the analyzer 2010 shown in FIG. 19, the sample cell 2048 is located toward the outer circumference of the centrifuge wheel 2050 (which can correspond, for example, to the sample cell holder 820 described herein). The sample cell 2048 preferably comprises windows that are substantially transmissive to energy in the sample beam E. For example, in implementations using mid-infrared energy, the windows may comprise calcium fluoride. The sample cell 2048 is in fluid communication with an injector system that permits filling the sample cell 2048 with a fluid sample (e.g., whole blood) and flushing the sample cell 2048 (e.g., with saline or a detergent). The injector system may disconnect after filling the sample cell 2048 with the fluid sample to permit free spinning of the centrifuge wheel 2050.

The centrifuge wheel 2050 can be spun by a centrifuge motor 2026. In some embodiments of the analyzer 2010, the fluid sample (e.g., a whole blood sample) is spun at a certain number of revolutions per minute (RPM) for a given length of time to separate blood plasma for spectral analysis. In some embodiments, the fluid sample is spun at about 7200 RPM. In some embodiments, the fluid sample is spun at about 5000 RPM or 4500 RPM. In some embodiments, the fluid sample is spun at more than one rate for successive time periods. The length of time can be approximately 5 minutes. In some embodiments, the length of time is approximately 2 minutes. In some embodiments, an anti-clotting agent such as heparin may be added to the fluid sample before centrifuging to reduce clotting. With reference to FIG. 19, the centrifuge wheel 2050 is rotated to a position where the sample cell 2048 intercepts the sample beam Eₛ, allowing energy to pass through the sample cell 2048 to the sample detector 2030.

The embodiment of the analyzer 2010 illustrated in FIG. 19 advantageously permits direct measurement of the concentration of analytes in the plasma sample rather than by inference of the concentration from measurements of a whole blood sample. An additional advantage is that relatively small volumes of fluid may be spectroscopically analyzed. For example, in some embodiments the fluid sample volume is between about 1 μL and 80 μL and is about 25 μL in some embodiments. In some embodiments, the sample cell 2048 is disposable and is intended for use with a single patient or for a single measurement.

In some embodiments, the reference detector 2036 and the sample detector 2030 comprise broadband pyroelectric detectors. As known in the art, some pyroelectric detectors are sensitive to vibrations. Thus, for example, the output of a pyroelectric infrared detector is the sum of the exposure to infrared radiation and to vibrations of the detector. The sensitivity to vibrations, also known as “microphonics,” can introduce a noise component to the measurement of the reference and sample energy beams Eᵣ, Eₛ using some pyroelectric infrared detectors. Because it may be desirable for the analyzer 2010 to provide high signal-to-noise ratio measurements, such as, e.g., S/N in excess of 100 dB, some embodiments of the analyzer 2010 utilize one or more vibrational noise reduction apparatus or methods. For example, the analyzer 2010 may be mechanically isolated so that high S/N spectroscopic measurements can be obtained for vibrations below an acceleration of about 1.5 G.

In some embodiments of the analyzer 2010, vibrational noise can be reduced by using a temporally modulated energy source 2012 combined with an output filter. In some embodiments, the energy source 2012 is modulated at a known source frequency, and measurements made by the detectors 2036 and 2030 are filtered using a narrowband filter centered at the source frequency. For example, in some embodiments, the energy output of the source 2012 is sinusoidally modulated at 10 Hz, and outputs of the detectors 2036 and 2030 are filtered using a narrow bandpass filter of less than about 1 Hz centered at 10 Hz. Accordingly, microphonic signals that are not at 10 Hz are significantly attenuated. In some embodiments, the modulation depth of the energy beam E may be greater than 50% such as, for example, 80%. The duty cycle of the beam may be between about 30% and 70%. The temporal modulation may be sinusoidal or any other waveform. In embodiments utilizing temporally modulated energy sources, detector output may be filtered using a synchronous demodulator and digital filter. The demodulator and filter are software components that may be digitally implemented in a processor such as the algorithm processor 416. Synchronous demodulators, coupled with low pass filters, are often referred to as “lock in amplifiers.”

The analyzer 2010 may also include a vibration sensor 2032 (e.g., one or more accelerometers) disposed near one (or both) of the detectors 2036 and 2030. The output of the vibration sensor 2032 is monitored, and suitable actions are taken if the measured vibration exceeds a vibration threshold. For example, in some embodiments, if the vibration sensor 2032 detects above-threshold vibrations, the system discards any ongoing measurement and “holds off” on performing further measurements until the vibrations drop below the threshold. Discarded measurements may be repeated after the vibrations drop below the vibration threshold. In some embodiments, if the duration of the “hold off” is sufficiently long, the fluid in the sample cell 2030 is flushed, and a new fluid sample is delivered to the cell 2030 for measurement. The vibration threshold may be selected so that the error in analyte measurement is at an acceptable level for vibrations below the threshold. In some embodiments, the threshold corresponds to an error in glucose concentration of 5 mg/dL. The vibration threshold may be determined individually for each filter 2015.

Certain embodiments of the analyzer 2010 include a temperature system (not shown in FIG. 19) for monitoring and/or regulating the temperature of system components (such as the detectors 2036, 2030) and/or the fluid sample. Such a temperature system can include temperature sensors, thermoelectrical heat pumps (e.g., a Peltier device), and/or thermistors, as well as a control system for monitoring and/or regulating temperature. In some embodiments, the control system comprises a proportional-plus-integral-plus-derivative (PID) control. For example, in some embodiments, the temperature system is used to regulate the temperature of the detectors 2030, 2036 to a desired operating temperature, such as 35 degrees Celsius.

The reference detector can be used to perform a check on the optical measurement path, for example, when saline, or another reference fluid is in the flow cell. For example, if a window on the flow cell were to become dirty, or light along the optical measurement path were otherwise obstructed, the measurement detector would see a reduced amount of light transmitted, while the reference detector would detect no reduction in light intensity. When the difference between light received by the measurement detector and the reference detector is different than an expected amount, the system can indicate that that optical system may have a problem. The reference detector can be used to perform a check on the light source. For example, if the reference detector detects a light intensity below a threshold value, or above a threshold value, or different than an expected value or range, or if the detected light intensity changes, the system can indicate that the light source may have a problem. In some embodiments, the system can use information from both the measurement detector and the reference detector in making the analyte concentration determinations. This can compensate for variations in the light source output. For example, if the light source outputs a reduced light intensity, the measurement detector can detect a corresponding reduced light intensity. If the system did not use information from the reference detector, the reduced light intensity measured by the measurement detector could result in an inaccurate analyte concentration determination. The reference detector can also detect a reduced light intensity, and the system can use make the analyte concentration determination based at least in part on the information from both detectors. In some cases a ratio or comparison between the light intensities detected by the measurement detector and the reference detector can be used in making the analyte concentration determination.

A fluid network interface 344 may allow the disposable system 304 to communicate with the network controller 336. This may allow one or more sensors within the fluid analysis system 300 to determine one or more measurements associated with the fluid sample. For example, the patient connector 320 may include a temperature sensor that identifies a temperature of the fluid sample near the source. In this way, for example, the fluid analysis system 300 can monitor (e.g., continuously, periodically, or intermittently) the temperature of the fluid in the patient 324. In some cases, a wire or wires can be embedded in a wall or walls of the tube, which can enable communication (or other electrical transfer) between the temperature sensor and the main system 308 (e.g., via the network or other controller). The system can monitor the temperature of the fluid (e.g., being drawn through the connector 320). Incorporating electrical leads, wires, or other elements configured for electrical power or other signal transmission can assist in allowing various fluidly-connected elements to also be electrically connected. Thus, a fluid connector that interfaces with a patient-connected line (e.g., a central-venous catheter or peripheral intravenous catheter) can be employed as (or can incorporate) a temperature sensor because it is closer to a patient's body and receives blood most directly and immediately from a patient when blood is drawn. After blood has been drawn for a given amount of time, it can have a warming effect on the connector or other surrounding structure. Initially, the monitored temperature can rise as fluid is drawn from the patient, and the temperature change can slow or stop after a time, indicating that the measurement temperature is approximately the same as the patient's body temperature. It can be helpful to insulate any electrical leads, metal, or other electrically conductive components from blood or fluid within a tube, to reduce risk that electrical current flows to or through a patient in harmful ways. It can also be useful to use low voltages and/or currents for safety and conservation reasons.

Additionally or alternatively, the network controller 336 may receive input from one or more bubble sensors and/or pressure sensors. These and other sensors described herein can be located in the disposable system 304, in the main system 308, or they can include components in both systems. In advantageous embodiments, a more expensive and long-lasting portion of such a sensor is located in the main system and less-expensive portions (and/or those configured for fluid contact) are positioned in the disposable system 304. In many cases The one or more bubble sensors can indicate whether a bubble has arrived within the fluid network 316. The one or more pressure sensors may provide a pressure reading at one or more points within the fluid network 316 (e.g., at tube junctions/intersections). Any bubble and/or pressure sensors included in the fluid analysis system 300 may share one or more features with those described above with reference to FIG. 1. The fluid network interface 344 may also allow communication between one or more enzymatic sensors (described below) and the main system 308. For example, the enzymatic sensor(s) may include electrical connection elements (e.g., pogo pins, etc.) to pass electrical signals to, e.g., the processor 332. An electrical current can be generated in a main analysis unit (e.g., using a battery, another DC, an A/C source, a transformer, a solar cell, etc.). This current can be transmitted to an enzymatic glucose biosensor feature. For example, an electrode can take up electrons needed to oxidize glucose and produce electric current. Some embodiments can result in a different measured electrical result depending on the amount of glucose present in a sample. Measured results (e.g., in the form of a current, a voltage, a resistance, etc.), can be transmitted to an analysis portion of a main instrument. This transmission can occur using the electrical connection elements described above. The disposable system 304 may include electrical circuitry to pass electrical signals among one or more elements of the disposable system 304 and/or main system 308.

The fluid analysis system 300 may be configured to measure the pressure in the fluid network 316 periodically, e.g., by using one or more pressure sensors in fluid communication with the fluid network 316. The at least one pressure sensor can monitor the pressure by measuring the pressure in the fluid handling network regularly (e.g., once every minute, once every thirty seconds, once every fifteen seconds, once every five seconds, once every two seconds, once every second, twice per second, three times per second, five times per second, ten times per second, etc.), on a substantially continuous basis, or by a single measurement.

A system interface 348 may be included in the fluid analysis system 300 to allow for communication between the disposable system 304 and the permanent system 308. For example, electrical communication may be initiated and maintained between the systems. Other types of communication (e.g., mechanical) are contemplated. For example, the system interface 348 may include various attachment mechanisms (e.g., snap fits, friction fits, locking features, adhesives, etc.). Electrical contact points for transferring or conveying current associated with an enzymatic sensor can be engineered to create a consistent, snug, reproducible, electrically sound connection. For example, these contact points can include one or more resilient members that exert force against other surfaces or objects. Sufficient electrical contact can be confirmed using a test current and an alarm can notify a user when electrical contact is inadequate. Mechanical resilience can be provided using metal springs or members biased using resilient rubber, plastic, composite, or other materials. Sliding or hinging contact can be created such that closing of a door, drawer, latch provides electrical contact. Any component (not just an enzymatic sensor) can be configured to incorporate electrical current such that electrical connection can benefit its function, for example, for diagnostic, control, monitoring, or troubleshooting purposes.

### Enzymatic Fluid Analysis

FIG. 3 schematically shows an example fluid analysis system 500 that may reflect many of the features described above with regard to the sampling and analysis system 400 and/or the fluid analysis system 300 above. The fluid analysis system 500 may include a patient connector 512, a flow cell 560, a blood sample detection sensor 554, one or more pumps 518, 535, and/or other features.

Fluid (e.g., blood) may be received through a patient connector 512 into the fluid analysis system 500. One or more pumps (e.g., a saline pump 535) may provide negative pressure within the fluid analysis system 500 to promote the intake of the fluid. The saline pump 535 may provide negative pressure in conjunction with the opening and closing of certain valves. For example, to draw fluid through the patient connector 512, a valve 540 (V6) may be closed while a valve 542 (V5) may remain open. Other valves 528, 532 (V1, V3) may be closed while the valves 526, 530 (V2, V4) may be opened. Each discrete fluid sample may be relatively small in order that a total volume of blood drawn may be reduced, thus preventing a need for blood transfusions into the patient. For example, the fluid analysis system 500 can be configured to draw between about 50 μL and 800 μL. The fluid analysis system 500 may preferably draw between about 100 μL and 350 μL in each sample. In some embodiments, the fluid analysis system 500 is configured to draw about 170 μL in each sample.

Advantageously the fluid analysis system 500 can be configured to return some or all of the sample to the patient. This can mitigate the need for blood transfusions to replace lost blood during the operation of the fluid analysis system 500. Moreover, this can reduce the need for disposing of the fluid, e.g., in a waste bladder or some other waste container. Hospitals can benefit from reducing costs and risks of biohazardous material disposal. Returning a larger portion of drawn blood can also allow for more frequent, or even steady or continuous blood withdrawal and analysis because the net blood loss may be zero or negligible. Advantageously the fluid analysis system 500 can be configured to return the fluid to the patient within a threshold time. The threshold time may be between about 20 seconds and 160 seconds. The fluid analysis system 500 may preferably return the fluid to the patient within a threshold time of between 45 seconds and 60 seconds. Fluid return to a patient can be especially useful if the fluid (e.g., blood) is treated gently while it is outside a patient. This can include limiting any additives (e.g., using innocuous substances such as saline) and avoiding inclusion of potentially harmful substances such as heparin. This can also include avoiding lysing, separation, coagulation, stacking, and the like. Gentle treatment can also include structures that reduce or minimize turbulence and improve laminar flow. The dimensions of any flow passages and/or flow cells can be large enough to allow blood particles to flow smoothly and avoid occlusions. Fluid flow can be generally steady to avoid stagnation and clotting, for example. In embodiments that do not centrifuge or lyse blood cells, for example, but rather filter, apply glucose-oxidase reactions, and/or apply electric voltages, the blood and any by-products of the measurement process can be returned to a patient.

The fluid sample may be returned to the patient in a number of ways. For example, the fluid analysis system 500 may be configured to close one or more valves, such as the valve 532, the valve 528, and the valve 540. A pump (e.g., the saline pump 535) may be configured to create a positive pressure on the fluid to push it back to the patient. The bubble sensor 516 and/or bubble sensor 517 may be configured to identify when the fluid sample has been successfully been returned to the patient or when it has successfully exited an apparatus en route to a patient. Saline solution may be injected into the patient between each sampling of the fluid. This can maintain an opening in a fluid source within the patient (e.g., the blood vessel).

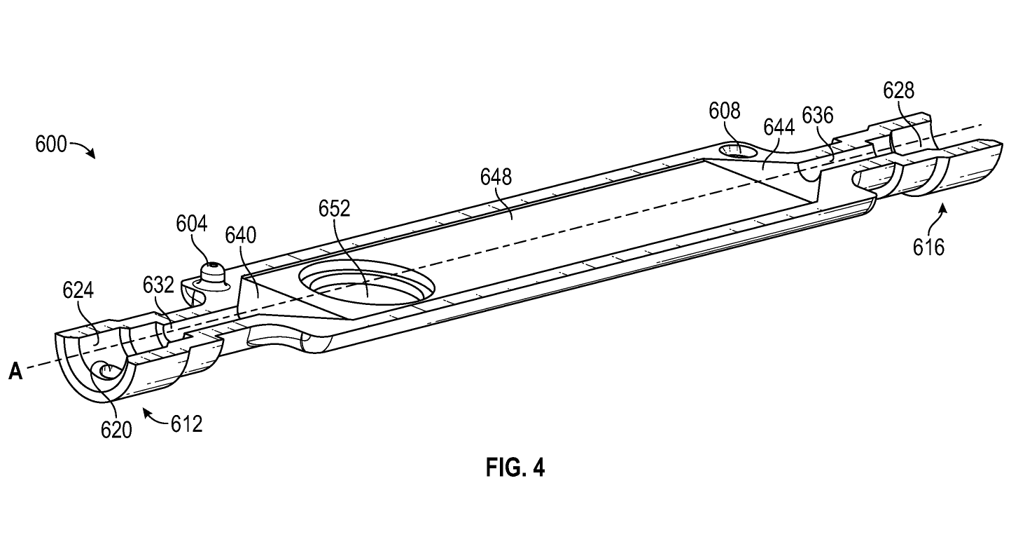

However, in some embodiments, portions of the sample that may be mixed with other materials or portions that are otherwise altered during the sampling and analysis process, or portions that, for any reason, are not to be returned to the patient, can also be placed in a waste bladder (not shown in FIG. 3). As fluid is drawn through an intake tube 536 (T1) of the fluid analysis system 500, the fluid may pass through one or more bubble sensors 516, 517 (BS11, BS12). As shown, two bubble sensors are included for redundancy purposes. However, one or fewer bubble sensors may be included. The one or more bubble sensors 516, 517 may be configured to detect the presence of a bubble in the fluid. While in some embodiments the presence of a bubble in the fluid sample may signal the arrival of a particular sample or “slug” of fluid, the bubble sensors 516, 517 may additionally or alternatively indicate that a bubble has inadvertently entered the fluid network of the fluid analysis system 500. As an additional example, the bubble sensors may indicate the presence of bubbles when returning the fluid to the patient. Thus, in some embodiments the fluid analysis system 500 can be disengaged, an alarm can sound, a pump can stop, or a flow can otherwise be impeded to prevent one or more bubbles from entering a patient. Such a bubble may indicate that system operation should be suspended until the bubble is removed from the fluid analysis system 500.While the valves 528, 532 are closed, fluid may be drawn through the valves 526, 530 and into the flow cell 560. The flow cell 560 may include one or more sensors or sensor elements, such as enzymatic sensors and/or optical cell components. The flow cell 560 may include a glucose sensor 568, a lactate sensor 564, and/or an optical cell 566. As shown, the fluid sample may pass first through the glucose sensor 568, then through the lactate sensor 564, and then into the optical cell 566. However, any order of these elements is possible (see, e.g., FIG. 4 below). The flow cell 560 may be configured such that one or more of the sensors (e.g., the glucose sensor 568, the lactate sensor 564) may be purchased from third-party suppliers and installed into the flow cell 560. The lactate sensor 564 and glucose sensor 568 may be disposed in a fluid series with one another and/or with the optical cell 566.

The optical cell 566 can be configured to interact with an optical interrogation device, not shown (e.g., in the optical system 328). The optical interrogation device can be configured to detect a presence of an interferent (e.g., hemoglobin (Hb)). The optical cell 566 can be configured to receive radiation (e.g., white light) therethrough. The fluid (e.g., blood) may absorb some of that radiation. The optical interrogation device can be configured to detect a transmission of radiation through the flow cell 560. The optical interrogation device can be configured to detect multiple aspects related to the interferent. For example, the optical interrogation device can be configured to detect various variants of hemoglobin or related interferents (e.g., oxyhemoglobin, deoxyhemoglobin, methemoglobin, carboxyhemoglobin, sulfhemoglobin, etc.). Absorption data can be obtained for one or more of the interferents and submitted to a processor (e.g., the processor 332). In some embodiments, the system can be configured to display one or more of these absorption data, such as in one or more absorption spectra. Other data may be displayed as well, such as one or more levels of the analytes detected as described herein. Some embodiments allow for measurement of Hemoglobin (Hb) in whole blood (blood that has not been separated, e.g., by centrifugation or lysing). Such measurement can occur using optical systems and methods, for example, with a device that measures red wavelengths and/or a wavelength corresponding to Hb. For example, a full spectrum spectrometer can be used with a broadband light source and absorption at multiple wavelengths can be used to determine an amount of total hemoglobin (tHb). For example, absorption at multiple wavelengths corresponding to different types of hemoglobin (e.g., oxyhemoglobin, deoxyhemoglobin, methemoglobin, carboxyhemoglobin, sulfhemoglobin, etc.) can be added combined or processed (optically and/or algorithmically) to determine tHb.

It may be advantageous to cause the flow of the fluid to occur at a particular rate. Exertion of fluid pressure on a system using a syringe pump, for example, can assist in achieving steady, rapid fluid flow. These conditions are often preferred to rapidly varying and/or slower flow rates. For example, optical measurements may be more reliable if the fluid flows at a rate that will allow the fluid (e.g., blood) to prevent non-ideal fluid conditions (e.g., nonlinear fluid effects, stacking of red blood cells). These effects can cause undue scatter and/or transmission of the radiation and result in unreliable or inaccurate measurement data. For example, the system can be configured to move fluid through the flow cell at a flow rate of 0.05 feet per second, 0.1 feet per second, 0.25 feet per second, 0.5 feet per second, 0.75 feet per second, 1 feet per second, 1.25 feet per second, 1.5 feet per second, 1.75 feet per second, 2 feet per second, or any values therebetween, or any ranges bounded by any combination of these values, although other flow rates can be used in some implementations. The system can be configured to provide a flow rate that varies by no more than 0.1 feet per second, 0.2 feet per second, 0.3 feet per second, 0.4 feet per second, 0.5 feet per second, 0.6 feet per second, 0.7 feet per second.

An optical system can be used for detection of blockages or abnormalities within a flowing system. An optical signal from a system trained on a flowing passage can typically fluctuate—for example, if the materials within the flowing fluid have varying shapes, densities, concentrations, and/or other optical properties. However, if this normal range of fluctuation stops and the signal becomes steady, this can indicate that a blockage has occurred and flow has slowed or stopped. A detected optical signal can also increase, which can indicate that red blood cells are stacking or otherwise clumping in a biological process such as clotting. Thus, optical outputs can be useful in diagnosing and assessing fluid flow in systems such as those described here. Thus, presence of an optical system can reduce the number of other sensors needed in an overall system because algorithms can be employed to accomplish more than one assessment function based on a single stream of data, for example. In FIG. 3, for example, a sensor 554 may be omitted if another optical system is programmed to perform its function or the equivalent. Useful optical systems can be configured such that light from a light source is purposefully diffused (e.g., by passing it through an opaque material or surface, by scratching or otherwise altering a surface of a transparent component). A diffusing component or feature can be included with a main instrument or an insertable (e.g., disposable) fluid-handling component such as a cartridge. Diffusing can help cause a more steady and predictable transmission of light through an optical flow cell and/or sample, for example. Diffusion can also attenuate output to avoid oversaturation of a detector. Diffusion can also result in optical averaging to avoid measurement and detection problems.

The glucose sensor 568 can be configured to detect a presence and/or level of glucose within the fluid. The glucose sensor 568 may comprise a glucose membrane and/or a glucose enzyme (e.g., glucose oxidase (GOx)¹⁵). The glucose membrane and/or glucose enzyme may be housed in a slot or receptacle within the flow cell 560. The slot can be configured to allow the fluid to pass over the glucose sensor 568 for a sufficient time to get an accurate reading of the glucose level in the fluid. However, it may also be advantageous to avoid allowing the fluid to slow or stop for too long in order to avoid negative effects in the fluid, such as clotting of blood. For example, the fluid analysis system 500 may be configured to allow the fluid sample to pass over the glucose sensor 568 for between about 1 and 8 seconds. In some embodiments, the fluid analysis system 500 is configured to allow the fluid sample to pass over the glucose sensor 568 for between 2 and 4 seconds. Sensors can have different times for saturation or other reading thresholds to be achieved. The glucose sensor 568 can be configured to allow plasma and glucose to pass through a membrane. Additionally or alternatively, the glucose sensor 568 can be configured to gather reliable measurements of a level of glucose within the fluid within a threshold time. The threshold time may be between 30 seconds and 90 seconds. In some embodiments, the threshold is one minute, such that measurements can be obtained in under one minute.

The lactate sensor 564 may be configured to detect a presence and/or level of lactate within the fluid. The detection and indication of lactate levels can provide many benefits for health care providers. Lactate levels may indicate whether a patient requires urgent medical attention. For example, higher than normal lactate levels can indicate the presence of a serious (even life-threatening) problem needing immediate intervention. The lactate sensor 564 may comprise a lactate membrane and/or a lactate enzyme (e.g., lactate oxidase (LOx)). The lactate membrane and/or lactate enzyme may be housed in a slot or receptacle within the flow cell 560. The slot can be configured to allow the fluid to pass over the lactate sensor 564 for a sufficient time to get an accurate reading of the glucose level in the fluid. The fluid analysis system 500 may be configured to allow the fluid sample to pass over the lactate sensor 564 for between about 1 and 8 seconds. In some embodiments, the fluid analysis system 500 is configured to allow the fluid sample to pass over the lactate sensor 564 for between 2 and 4 seconds (or within another acceptable range). The lactate sensor 564 can be configured to allow plasma and lactate to pass through the glucose membrane. Additionally or alternatively, the lactate sensor 564 can be configured to gather reliable measurements of a level of glucose within the fluid within a threshold time. The threshold time may be between 20 seconds and 120 seconds. In some embodiments, the threshold is one minute, such that measurements can be obtained in under one minute.

The flow cell 560 may comprise an optically transmissive portion. The flow cell 560 may be formed of an optically transmissive material, such as a plastic or other polymer (e.g., TOPAS® COC plastic). The flow cell 560 may comprise a portion that can be in optical communication with an optical interrogation system (e.g., the optical system 328 of FIG. 2).

The fluid analysis system 500 can include a blood sample detection sensor 554. The blood sample detection sensor 554 can be disposed in fluid series with the flow cell 560. As shown, the blood sample detection sensor 554 is disposed such that the fluid reaches the blood sample detection sensor 554 after it passes through the flow cell 560.

Advantageously the enzymatic fluid analysis system 500 can include a flow path extension 550. The flow path extension 550 can allow the enzymatic fluid analysis system 500 to draw fluid steadily and/or continuously. This can help prevent various problems during sampling of the fluid (e.g., blood clotting, nonlinear fluid dynamic issues). Additionally or alternatively, the flow path extension 550 can permit the steady draw of the fluid without allowing the fluid sample to flow beyond a valve 542 and/or a connector C2. This can prevent blood from entering a tube configured only for saline (e.g., saline pump tube 580). The flow path extension 550 can comprise tubing of a threshold length. The threshold length can be between about 5 feet and 45 feet. In some embodiments, the threshold length is about 20 feet. For example, 20 feet has been found to be particularly advantageous in certain embodiments to allow for the benefits described above while minimizing excess tubing. The inner diameter of the tubing can be between 500 μm and 1800 μm. In some embodiments, the inner diameter is about 1000 μm.

The fluid analysis system 500 may further include a saline source 546 and/or a saline pump 535. At the start of a measurement cycle, various lines, including a saline source tube 578, a flow cell tube 582, the flow cell 560, a delivery tube 584, and an intake tube 536 can be filled with saline that can be introduced into the system through the saline source tube 578, and which can come from the saline source 546 and/or drawn using a saline pump 535. The saline pump 535 and/or the saline source 546 can be provided separately from the fluid analysis system 500. For example, a hospital can use existing saline bags and infusion pumps to interface with the described system. Alternatively, the saline pump 535, for example, may be formed as part of the fluid analysis system 500.

In some embodiments, it may be advantageous to control a level of an analyte (e.g., glucose) in a patient using an embodiment of the fluid analysis system 500 described herein. Although certain examples of glucose control are described below, embodiments of the systems and methods disclosed herein may be used to monitor and/or control other analytes (e.g., lactate).

For example, diabetic individuals control their glucose levels by administration of insulin. If a diabetic patient is admitted to a hospital or ICU, the patient may be in a condition in which he or she cannot self-administer insulin. Advantageously, embodiments of the analyte detection systems disclosed herein may be used to control the level of glucose in the patient. Additionally, it has been found that a majority of patients admitted to the ICU exhibit hyperglycemia without having diabetes. In such patients it may be beneficial to monitor and control their blood glucose level to be within a particular range of values. Further, it has been shown that tightly controlling blood glucose levels to be within a stringent range may be beneficial to patients undergoing surgical procedures.

A patient admitted to the ICU or undergoing surgery may be administered a variety of drugs and fluids such as Hetastarch, intravenous antibiotics, intravenous glucose, intravenous insulin, intravenous fluids such as saline, etc., which may act as interferents and make it difficult to determine the blood glucose level. Moreover, the presence of additional drugs and fluids in the blood stream may require different methods for measuring and controlling blood glucose level. Also, the patient may exhibit significant changes in hematocrit levels due to blood loss or internal hemorrhage, and there can be unexpected changes in the blood gas level or a rise in the level of bilirubin and ammonia levels in the event of an organ failure. Embodiments of the systems and methods disclosed herein advantageously may be used to monitor and control blood glucose (and/or other analytes) in the presence of possible interferents to estimation of glucose and for patients experiencing health problems.

In some environments, Tight Glycemic Control (TGC) can include: (1) substantially continuous monitoring (which can include periodic monitoring, at relatively frequent intervals of every 1, 5, 15, 30, 45, and/or 60 minutes, for example) of glucose levels; (2) determination of substances that tend to increase glucose levels (e.g., sugars such as dextrose) and/or decrease glucose levels (e.g., insulin); and/or (3) responsive delivery of one or more of such substances, if appropriate under the controlling TGC protocol. For example, one possible TGC protocol can be achieved by controlling glucose within a relatively narrow range (for example between 70 mg/dL to 110 mg/dL). As will be further described, in some embodiments, TGC may be achieved by using an analyte monitoring system to make continuous and/or periodic but frequent measurements of glucose levels.

In some embodiments, the analyte detection system schematically illustrated in FIG. 3 may be used to regulate the concentration of one or more analytes in the sample in addition to determining and monitoring the concentration of the one or more analytes. In some cases, the analyte detection system may be used in an ICU to monitor (and/or control) analytes that may be present in patients experiencing trauma. In some implementations, the concentration of the analytes is regulated to be within a certain range. The range may be predetermined (e.g., according to a hospital protocol or a physician's recommendation), or the range may be adjusted as conditions change.

In an example of glycemic control, a system can be used to determine and monitor the concentration of glucose in the sample. If the concentration of glucose falls below a lower threshold, glucose from an external source can be supplied. If the concentration of glucose increases above an upper threshold, insulin from an external source can be supplied. In some embodiments, glucose or insulin may be infused in a patient continuously over a certain time interval or may be injected in a large quantity at once (referred to as “bolus injection”).

In some embodiments, a glycemic control system may be capable of delivering glucose, dextrose, glycogen, and/or glucagon from an external source relatively quickly in the event of hypoglycemia. As discussed, embodiments of the glycemic control system may be capable of delivering insulin from an external source relatively quickly in the event of hyperglycemia.

Returning to FIG. 3, this figure schematically illustrates an embodiment of a fluid handling system that comprises an optional analyte control subsystem. The analyte control subsystem may be used for providing control of an analyte such as, e.g., glucose, and may provide delivery of the analyte and/or related substances (e.g., dextrose solution and/or insulin in the case of glucose). The analyte control subsystem can comprise a insulin source 548 such as, for example, the analyte (or a suitable compound related to the analyte) dissolved in water or saline. The insulin source 548 can be coupled to an insulin pump 518. The insulin source 548 and the insulin pump 518 can be provided separately from the analyte control subsystem. For example, a hospital advantageously can use existing bags and pumps with the subsystem.

As schematically illustrated in FIG. 3, the insulin source 548 is in fluid communication with the patient connector 512 via an insulin source tube 576 and suitable connectors. A pinch valve (e.g., the valve 528)may be disposed adjacent the insulin source tube 576 to regulate the flow of fluid from the insulin source 548. A patient injection port can be located at a short distance from the proximal port of the central venous catheter or some other catheter connected to the patient.

In an example implementation for glycemic control, if the analyte detection system determines that the level of glucose has fallen below a lower threshold value (e.g., the patient is hypoglycemic), a control system (e.g., the fluid system controller 405 in some embodiments) controlling an infusion delivery system may close the pinch valve to prevent infusion of insulin and/or saline into the patient. The control system may open the pinch valve 2786 and insulin solution from the insulin source 548 can be infused (or alternatively injected as a bolus) into the patient. After a suitable amount of solution has been infused to the patient, the pinch valve can be closed. In some systems, the amount of solution for infusion (or bolus injection) may be calculated based on one or more detected concentration levels of glucose. [0276] If the analyte detection system determines that the level of glucose in the fluid (e.g., by detection by the flow cell 560) has increased above an upper threshold value (e.g., the patient is hyperglycemic), the control system may prevent infusion of saline into the patient. The control system may open the pinch valve (e.g., the valve 528), and insulin can be infused (or alternatively injected as a bolus) into the patient. After a suitable amount of insulin has been infused (or bolus injected) to the patient, the control system can close the pinch valve and open the valve 526 and/or the valve 530 to allow flow of saline. The suitable amount of insulin may be calculated based on one or more detected concentration levels of glucose in the patient. The insulin source 548 advantageously may be located at a short enough fluidic distance from the patient such that insulin can be delivered to the patient within about one to about ten minutes. In other embodiments, the insulin source 518 may be located at the site where the patient tube 512 interfaces with the patient so that insulin can be delivered to the patient within about one minute.

In some embodiments, sampling bodily fluid from a patient and providing medication to the patient may be achieved through the same lines of the fluid handling system. For example, in some embodiments, a port to a patient can be shared by alternately drawing samples and medicating through the same line. In some embodiments, a bolus can be provided to the patient at regular intervals (in the same or different lines). For example, a bolus of insulin can be provided to a patient after meals. In embodiments comprising a shared line, a bolus of medication can be delivered when returning part of a body fluid sample back to the patient. In some implementations, the bolus of medication is delivered midway between samples (e.g., every 7.5 minutes if samples are drawn every 15 minutes). In other embodiment, a dual lumen tube can be used, wherein one lumen is used for the sample and the other lumen to medicate. In yet another embodiment, an analyte detection system (e.g., an “OptiScanner®” monitor) may provide suitable commands to a separate insulin pump (on a shared port or different line).

FIGS. 4-15 illustrate various aspects of an example embodiment of a flow cell 560 that can be included in the fluid analysis system 500. FIG. 4 shows an isometric view of an example fluid cell component 600 that can be used to construct a flow cell. A flow cell may be comprised of one or more fluid cell components 600. These fluid cell components 600 can allow for reducing a number of flow cell components that are required for manufacture. For example, two fluid cell components 600 may be attached (e.g., laser welded) to one another to create an enclosed (e.g., complete) flow cell (see, e.g., FIGS. 8-15). The fluid cell component 600 can represent half of an enclosed flow cell.

The fluid cell component 600 can define an axis A as shown. The axis A may run from a first end 612 to a second end 616. The fluid cell component 600 can include a first approach 624 and a second approach 628 opposite the first approach 624. The approaches 628, 624 may include an inner radius (as measured perpendicular to the axis A) that is greater than an inner radius of corresponding inlets/outlets 632, 636. Fluid may flow from the first approach 624 into the first inlet/outlet 632. From the first inlet/outlet 632, the fluid sample may pass through a first transition portion 640 into a main cell chamber 648. The main cell chamber 648 may include a first slot 652. As described in more detail herein, the first slot 652 may be configured to house an enzymatic sensor (e.g., glucose sensor, lactate sensor).

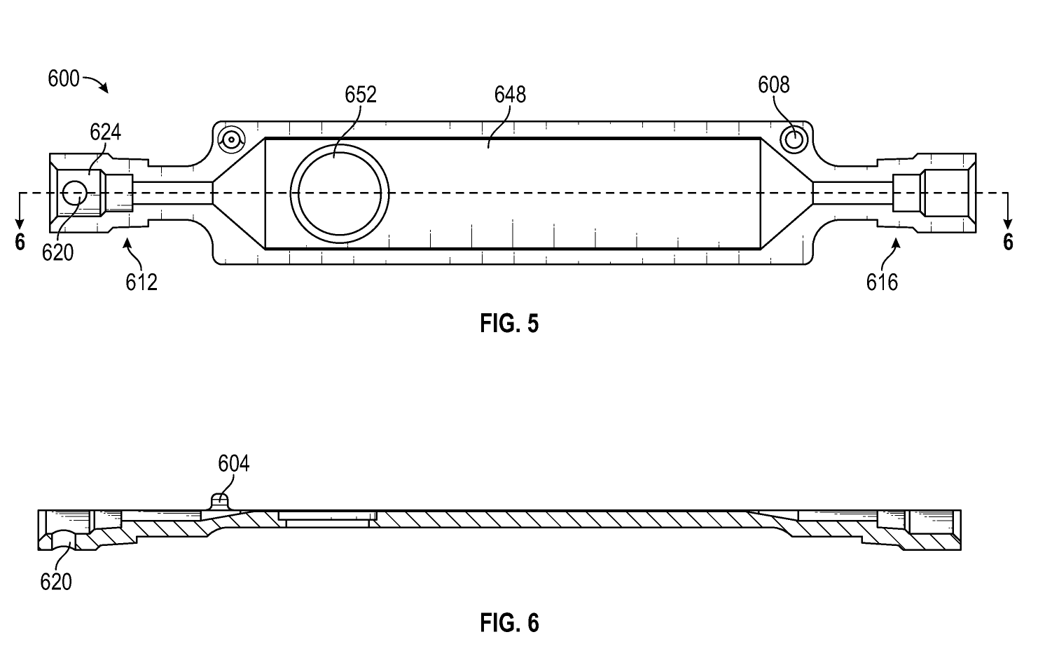

The fluid cell component 600 can include various features to aid in assembling the final flow cell. For example, a protruding portion 604 can be included within a body of the flow cell and/or outside a portion where fluid may be configure to flow. The protruding portion 604 can be configured to be inserted into a corresponding receiving portion 608 of a second fluid cell component 600. This connection may be a friction attachment, an adhesive attachment, a snap fit, and/or other means for attaching the two fluid cell components 600 together. A first adhesive inlet 620 can be configured to allow for the insertion of adhesive between two fluid cell components 600, as is discussed in more detail below. The fluid cell component 600 can be molded and/or may comprise an optically transmissive material (e.g., plastic or other compound). FIG. 5 shows a top view of the fluid cell component 600 shown in FIG. 4. FIG. 6 shows a cross section of the embodiment of FIG. 5 along the axis A. FIG. 7 shows an isometric view of the fluid cell component 600 from below.

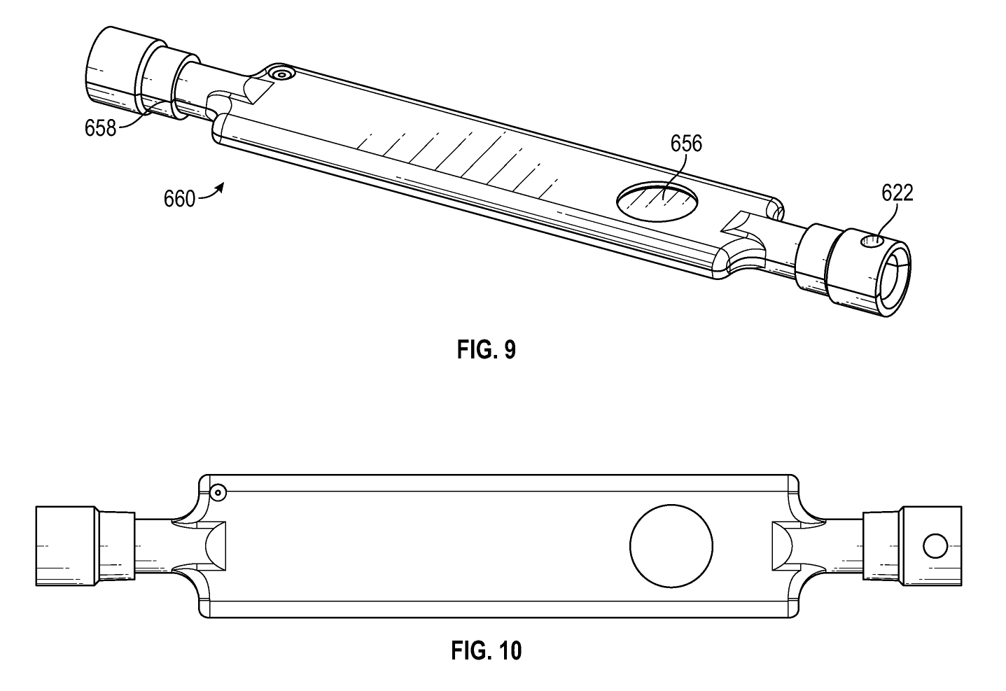

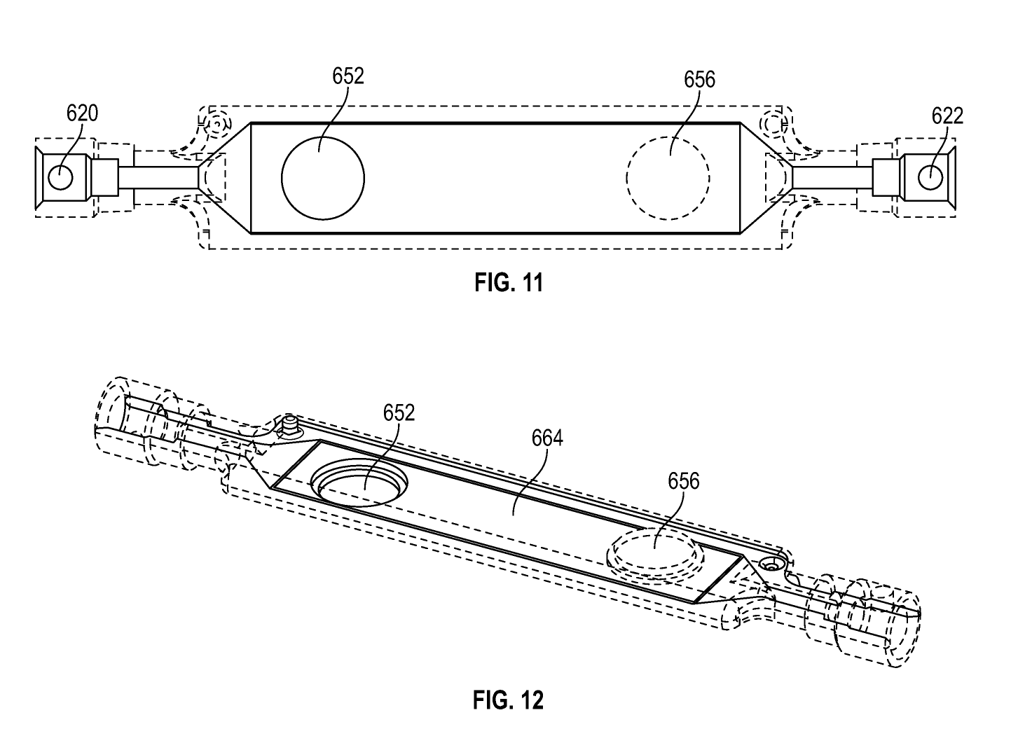

FIGS. 8-13 show how two cell components (e.g., fluid cell components 600) may be attached to create an enclosed assembled fluid cell 660. FIG. 8 shows an isometric view of a solid lower fluid cell component 600 and a translucent wireframe upper fluid cell component 600. The assembled fluid cell 660 shows a first slot 652 and a second slot 656. As shown, the first slot 652 can be included in the first fluid cell component 600 and the second slot 656 can be included in the second fluid cell component 600. One or more of the first slot 652 and/or second slot 656 can be configured to house a corresponding enzymatic sensor, as described above. FIG. 9 shows the embodiment of FIG. 8 where including a solid upper fluid cell component 600. FIG. 10 shows a bottom solid view of the embodiment described in FIG. 8. FIG. 11 shows a translucent bottom view of the embodiment of FIG. 8. The relative locations of the first slot 652 and second slot 656 can be seen. Moreover, relative locations of the first adhesive inlet 620 and the second adhesive inlet 622 can be seen.

FIG. 12 shows an isometric view of a solid lower fluid cell component 600 and a translucent upper fluid cell component 600. A location of an optical investigation portion 664 is shown. The optical investigation portion 664 may correspond to the optical cell 566 discussed above. The optical investigation portion 664 can be configured for optical communication with an optical interrogation device (e.g., laser, LED). The optical investigation portion 664 can be configured to allow radiation (e.g., white light) to pass therethrough and interact with a fluid sample. In this way, the optical interrogation device (e.g., part of the optical system 328) can detect various aspects of the fluid.

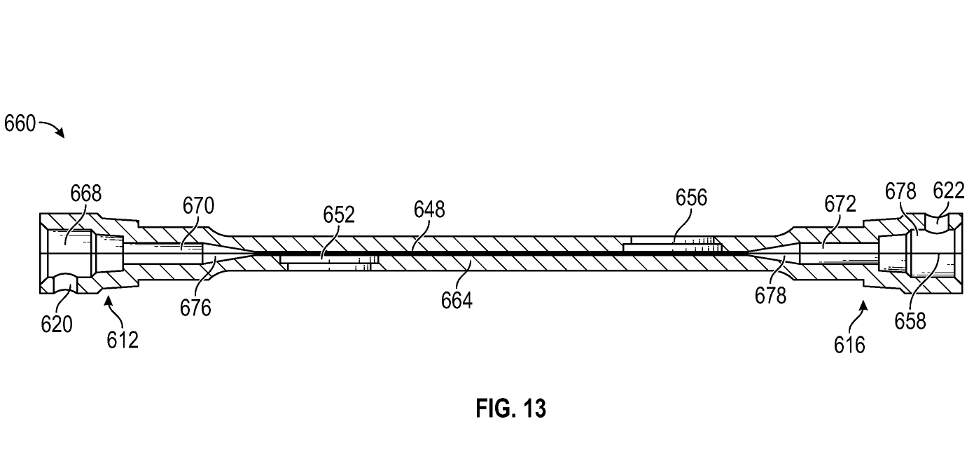

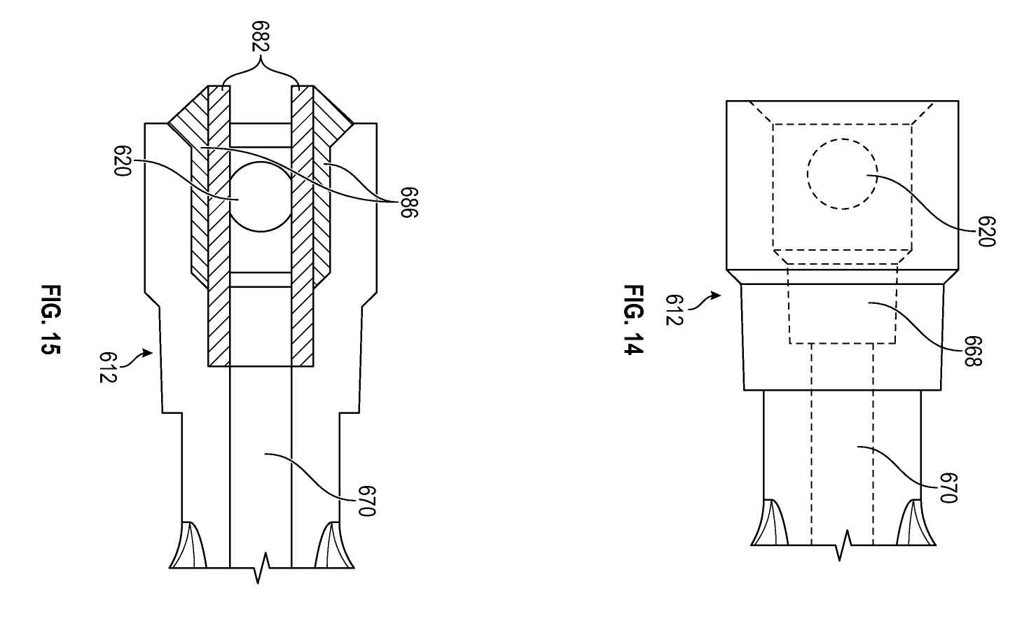

FIG. 13 shows a cross section view of the assembled fluid cell 660 shown in FIG. 8. The assembled fluid cell 660 can include a first end 612 and a second end 616. The first end 612 of the assembled fluid cell 660 can include a first tube interface 668, a fluid inlet 670, and/or a first fluid profile modifier 676. The first end 612 may advantageously include a first adhesive inlet 620. The second end 616 may include a second fluid profile modifier 678, a fluid outlet 672, and/or a second fluid profile modifier 678. The second end 616 may also include a second adhesive inlet 622. The assembled fluid cell 660 may also include a main cell chamber 648 that is in fluid communication with the first slot 652 and/or the second slot 656. Moreover, the main cell chamber 648 may comprise the optical investigation portion 664. The first tube interface 668, the fluid outlet 672, the first adhesive inlet 620, and the second adhesive inlet 622 are described in more detail below with regard to FIGS. 14-15.

The assembled fluid cell 660 can be configured to receive the fluid sample through the fluid inlet 670. An inner diameter of the fluid inlet 670 can be configured to be the same as an inner diameter of one or more tubes in the system (e.g., the flow cell tube 582, the delivery tube 584 of FIG. 3). The inner diameter of the fluid inlet 670 can be the same as an inner diameter of the fluid outlet 672. The inner diameter of the fluid inlet 670 and/or the fluid outlet 672 can be between 500 μm and 1800 μm. In some embodiments, the inner diameter is about 1000 μm. A cross section of the fluid inlet 670 may be circular or elliptical.

Flow of the fluid can pass from the fluid inlet 670 to the first fluid profile modifier 676. The first fluid profile modifier 676 can have a constant cross-sectional area at one or more points along axis A. In some cases, the first fluid profile modifier 676 can have a constant cross-sectional area along a majority of, or along the full length of, the profile modifier 676. A constant cross-sectional area as described can mitigate irregular flow patterns (e.g., turbulent flow, clotting, stacking of red blood cells) as the fluid transitions in to the main cell chamber 648. The fluid outlet 672 may include one or more features common with the fluid inlet 670 described above. Additionally or alternatively, the second fluid profile modifier 678 may include one or more features common with the first fluid profile modifier 676 described above.

The main cell chamber 648 may include two enzymatic sensors (e.g., the glucose sensor 568, the lactate sensor 564). Each of the enzymatic sensors may be disposed within corresponding slots 652, 656. These slots may be configured to allow fluid flow over each sensor. The sensors can be disposed to allow passage of fluid (e.g., blood plasma, the fluid sample) to pass (e.g., diffuse) along a direction perpendicular to the axis A and/or perpendicular to a direction of fluid flow through the assembled fluid cell 660. The main cell chamber 648 can also include an optical investigation portion 664. In some embodiments, the optical investigation portion 664 comprises a body of the assembled fluid cell 660 through which radiation (e.g., white light) can pass. In some designs, the optical investigation portion 664 comprises an optically transparent and/or rigid material. The cross-sectional area of the main cell chamber 648 can the same cross-sectional area as the inlet 670, and/or as the profile modifier 676. An interior of the flow cell 660 can have a constant cross-sectional area along a majority of, or along the full length of, the flow cell 660 along the axis A. The main cell chamber 648 can height of 50 microns, 75 microns, 100 microns, 125 microns, 150 microns, 175 microns, 200 microns, 225 microns, 250 microns, or any values therebetween, or any ranges bounded by any combination of these values, although other heights can also be used. The main cell chamber 648 can have a width of 1000 microns, 2000 microns, 3000 microns, 4000 microns, 5000 microns, 6000 microns, 7000 microns, 8000 microns, 9000 microns, or 10,000 microns, or any values therebetween, or any ranges bounded by any combination of these values, although others could be used. The flow cell 660 can have features that are the same as or similar to the flow cell embodiments disclosed herein.

In some cases, variations in manufacturing can cause slight differences in dimensions or other attributes of the system. In some embodiments, the system can include an attribute indicator that is indicative of an attribute of the system. The attribute can include an optical path length, one or more dimension of the flow cell, a measured height of the main cell chamber of the flow cell, etc. The attribute indicator can include a Quick Response Code (QR code), a bar code, an Radio-frequency identification (RFID), etc.

The assembled fluid cell 660 may be included as part of a disposable system described above. This process may include supplying adhesive (e.g., glue) to a portion of the assembled fluid cell 660 and/or attaching one or more tubes thereto. FIGS. 14-15 show a detail view of a cross section of the first end 612 of the assembled fluid cell 660. FIG. 14 shows the first end 612 before the insertion of adhesive and/or tubing therein. The first tube interface 668 may include one or more portions having respective inner diameters. These can be beneficial in allowing the attachment (e.g., adhering) of a tube of the system therein. FIG. 15 shows the first end 612 of the assembled fluid cell 660 after insertion of a tube and adhesive. A cross section of the tube is shown by tube walls 682. The tube walls 682 may define an inner diameter identical or similar to that of the fluid inlet 670. After the tube walls 682 have been placed within the first tube interface 668 (e.g., abutting the first tube interface 668), adhesive 686 may be supplied through the first adhesive inlet 620. The resulting adhesive 686 profile is schematically shown. The adhesive 686 may be configured to fill remaining gaps between the tube walls 682 and the assembled fluid cell 660.

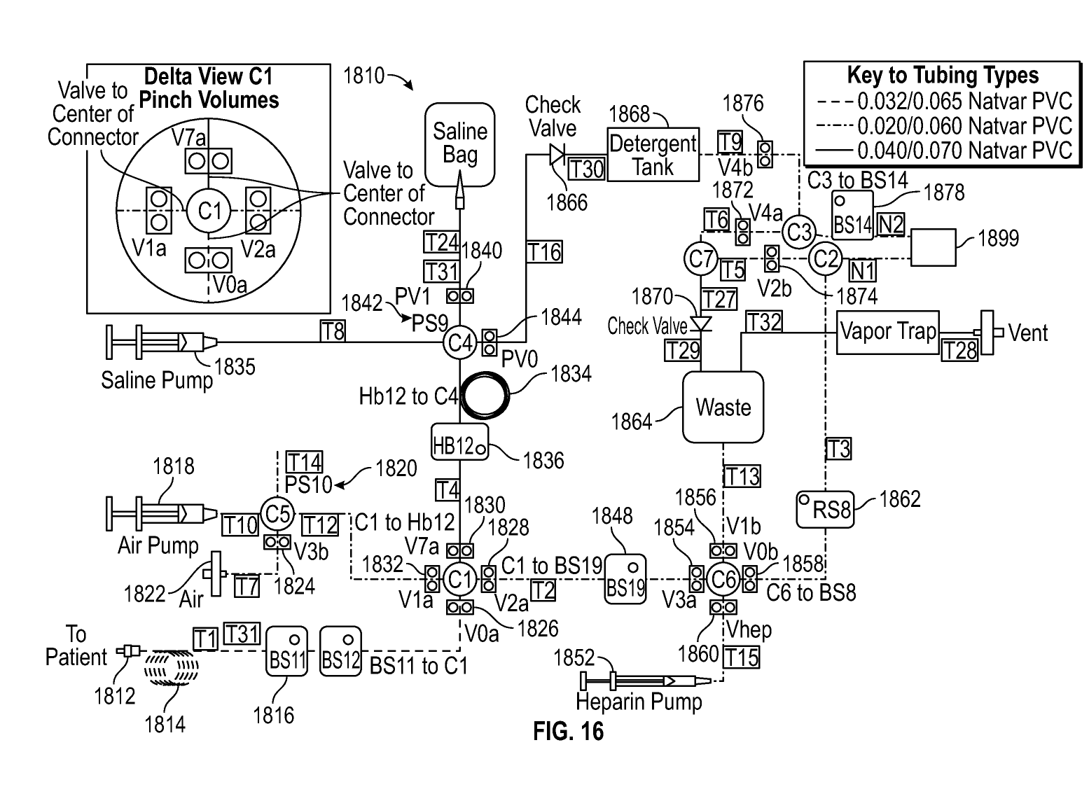

FIG. 16 schematically depicts a fluid system for periodically drawing precise amounts of fluid containing analytes into an analysis system. A fluid handling and analysis system can measure additional analytes using the system and apparatus described above. Moreover, the general structure described above can be modified or otherwise configured to incorporate additional sensors and/or measurement structures. For example, additional optical sources and spectrometers can be added to the same general structure described herein. The example described below can apply or be modified to enhance approaches for measuring other analytes, can apply to other wavelengths, other enzymatic reactions, other measurement modalities, including optical and non-optical modalities, etc.

Various embodiments disclosed hereby can relate to the electrochemical, enzymatic, spectroscopic (e.g., near infrared) or other measurement of one or more analytes in a bodily fluid (e.g., blood) of a patient. The bodily fluid can be drawn out of the patient and positioned within the system for processing and/or analysis as discussed herein.

An analyte sensor can be used in a fluid-handling system with various configurations. FIG. 4 depicts one such generalized fluid-handling system 404. In this fluid-handling system context, FIG. 16 schematically illustrates the layout of an example embodiment of a fluid system 1810. This embodiment may be used in part or as a whole in conjunction with or as an alternative to the example embodiment fluid systems shown in FIG. 3.

At the start of a measurement cycle, most tubing can be filled with saline that can come from a saline bag 1846. The saline bag 1846 can be provided separately from the system 1810. For example, a hospital can use existing saline bags and/or saline pumps to interface with the described system. Before drawing a bodily fluid sample, the saline in part of the system 1810 can be replaced with air 1822. Thus, for example, the following valves can be closed: valve 1844 (PV0), 1840 (PV1), 1826 (V0a), 1860 (Vhep), and 1874 (V2b). At the same time, the following valves can be open: valves 1832 (V1a), 1828 (V2a), 1854 (V3a), 1858 (V0b), 1824 (V3b), and 1872 (V4a). An air pump 1818 pumps air through the system, pushing saline through the system into a waste bag 1864.

Next, a sample can be drawn. With valves 1832 (V1a), 1840 (PV1), 1844 (PV0), and 1828 (V2a) closed, a saline pump 1835 is actuated to draw sample fluid to be analyzed (e.g., blood) from a fluid source (e.g., a laboratory sample container, a living patient, etc.). The sample fluid is drawn up into the patient tubing, though the tube past the two flanking portions of the open pinch-valve 1826 (V0a), through the first connector C1, into the looped tube 1834, and past the arrival sensor 1836 (Hb12). The arrival sensor 1836 (Hb12) may be used to detect the presence of blood in the tube.

The system 1810 can measure the pressure in the fluid-handling network, e.g., by using one or more pressure sensors, such as pressure sensor 1842 (PS9) and/or pressure sensor 1820 (PS10). The one or more pressure sensors can be in fluid communication with the fluid-handling network. The at least one pressure sensor can monitor the pressure, e.g., by measuring the pressure in the fluid handling network periodically (e.g., once every minute, once every thirty seconds, once every fifteen seconds, once every five seconds, once every two seconds, once every second, twice per second, three times per second, five times per second, ten times per second, etc.), on a substantially continuous basis, or by a single measurement.

Before drawing the sample, the tubes can be filled with saline and the hemoglobin level (Hb) is zero. The tubes that are filled with saline are in fluid communication with the sample source at 1812. When the saline is drawn toward the saline pump 1835, the fluid meant to be analyzed is also drawn into the system because of suction forces in the closed fluid system. The saline pump 1835 draws a relatively continuous column of fluid through the system, from tubing loop 1814, past bubble sensor 1816 (BS11), past first connection C1, and past arrival sensor 1836 (Hb12). The fluid column first comprises generally nondiluted saline, then a mixture of saline and sample fluid, then eventually minimally diluted and/or nondiluted sample fluid. The arrival sensor 1836 (Hb12) can detect and/or verify the presence of blood in the tubes. In some embodiments, when the sensed hemoglobin level reaches some pre-set value, substantially undiluted blood is present at first connection C1. The loop of tubing 1834 can provide additional length to the tubing. The additional length makes it more likely that a sufficient nondiluted portion of the fluid has reached the tubing. The system can include a plurality of arrival sensors (e.g., bubble sensor 1816 (BS11), bubble sensor 1848 (BS19), arrival sensor 1862 (BS8), bubble sensor 1878 (BS14), and arrival sensor 1836 (HB12), which can be configured to detect the arrival of a fluid (e.g., bodily fluid such as blood) at a plurality of locations in the fluid handling network.

If nondiluted and/or minimally diluted blood is present at the first connector C1, a sample can be directed toward a measurement portion. Bubble sensor 1848 (BS19) can detect the arrival of the sample. The sample can be directed into a flow cell, which can comprise part of an analysis portion 1899, which can comprise one or more sample conditioning devices (e.g., a lysing apparatus) and one or more sample measurement devices (e.g., an optical and/or enzymatic sensor or series of sensors). The sample can start out as a whole blood sample. A series of pinch valves, valves 1854 (V3a) and 1828 (V2a), can be closed in order to a channel which can form part of the analysis portion 1899. The analysis portion can include a flow cell 560 similar to the one described with respect to FIG. 3. The valves can constrain the flow rate at each end of the analysis portion 1899 and can fill a flow cell channel. In some embodiments, the flow cell channel can have entry and exit points that are smaller in cross-sectional area than the connecting tubes, while the center of the flow cell channel is larger in cross-sectional area than the connecting tubes. In some embodiments, the contained volume of the flow cell can approximate the cross-sectional area of the connecting tubes. In some embodiments, the contained volume may be within 10% of the cross-sectional area of the connecting tubes.

A flow cell can be used in conjunction with a near and/or mid infrared light source and a near and/or mid infrared spectrometer. The spectrometer can measure a variety of parameters. For example, the near infrared spectrometer may measure oxygen, hemoglobin, CO₂, pH, lactate, fluid flow rate, presence of fluid, color of fluid, etc. Energy from the infrared source can pass through the center of a flow cell before being measured by the near infrared spectrometer.

Before or after the near infrared spectrometer has taken measurements of the sample, the sample can be heparinized. An amount of anticoagulant (e.g., heparin) can be introduced by the syringe-style heparin pump 1852. A series of pinch valves, valves 1860 (Vhep) and 1858 (V0b) can be open, while valve 1854 (V3a) can be closed to prevent anticoagulant from mixing with the unused fluid. The heparin components may be separate from the fluid system 1810. Heparin pinch valve 1860 (Vhep) can be closed to prevent flow from or to the heparin pump 1852, and a heparin waste pinch valve 1856 (V1b) can be closed to prevent flow from or to the waste container 1864 from this junction. When bubble sensor 1862 (BS8) indicates the presence of a sample, the unused bodily fluid can be returned to its source. The saline pump 1835 pushes the fluid back to the patient by opening the valve 1826 (V0a), closing the valves 1832 (V1a) and 1828 (V2a), and keeping the valve 1830 (V7a) open. Saline from the saline bag 1846 may then flush the tubing.

Following the return of the unused fluid, the heparinized sample can be pushed through connector C2 into a sample cell (which can be part of an analysis portion 1899). Pump movement and valve position corresponding to each stage of fluid movement can be coordinated by one or more multiple controllers, such as the fluid system controller 405 of FIG. 1. The sample may be divided into multiple blood slugs, each separated by air bubbles injected into the system. Bubble sensor 1878 (BS14) may detect the leading edge as well as the end of each slug. Air pump 1818 may stop forcing the fluid column through the tubing when the bubble sensor 1878 (BS14) has detected one or more leading slugs that can serve to flush any residual saline out of the sample cell. One slug can be left in the sample cell and measured using mid-infrared spectroscopy (e.g., for glucose, lactate, or other analyte concentration). The leading slugs can be deposited in the waste container 1864 by passing through open pinch valve 1872 (V4a), while valve 1874 (V2b) may be closed. After analysis is complete, the sample slug can also be deposited in the waste container 1864. A check valve 1870 prevents the sample from exiting the waste container 1864. In some advantageous embodiments, air bubbles are not injected and separate slugs are not created.

In some embodiments, following analysis of the sample, the sample may be flushed by air pump 1818 (or a saline pump) and sent to the waste container 1864. Cleaner (e.g., a detergent such as tergazyme A) may flow through and clean the tubing surrounding the sample cell. Detergent tank 1868 may provide cleaner that is flushed through the tubing leading to and from the analysis portion 1899 and into the waste container 1864. Check valve 1866 prevents cleaner from flowing from the detergent tank toward the saline bag 1846 and saline pump 1835. Following the cleansing flush, saline can be drawn from the saline bag 1846 for a second flush. The saline pump 1836 can flush the cleaning solution out using the saline. This saline flush pushes saline through the tubing past the arrival sensor 1836 (Hb12), the heparin pump 1852, the analysis portion 1899 and into the waste container 1864. In some embodiments, the following valves are open for this flush: 1830 (V7a), 1828 (V2a), 1872 (V4a), and the following valves are closed: 1826 (V0a), 1832 (V1a), 1874 (V2b), and 1876 (V4b).

When the fluid source is a living entity such as a patient, a low flow of saline (e.g., 1-5 mL/hr) is preferably moved through the tubing 1812 and 1814 and into the patient to keep the patient's vessel open (e.g., to establish a keep vessel open, or “KVO” flow). This KVO flow can be temporarily interrupted when fluid is drawn into the fluid system 1810. The source of this KVO flow can be the saline pump 1835 or the air pump 1818. Preferably, the time between measurement cycles is longer than the measurement cycle itself (for example, the time interval can be longer than ten minutes, shorter than ten minutes, shorter than five minutes, longer than two minutes, longer than one minute, etc.).

Numerous sensors, fluid lines, pinch valves, junctions, pumps, fluid sources, and fluid receptacles are shown, largely consistent with the disclosure herein describing other fluidics diagrams (e.g., FIG. 3). An analysis portion 1899 can include a flow cell, such as any flow cell described herein. This analysis portion can comprise a sample conditioning device (e.g., a lysing apparatus) and a sample measurement device (e.g., an optical and/or enzymatic sensor or series of sensors). A lysing apparatus can be helpful to break down red blood cells to help avoid stacking or other clumping of red blood cells that often occurs when a blood flow rate reduces or when blood is disturbed, removed from a body, or has its flow curtailed. Lysing can also assist in releasing analytes from being biologically bound or contained, making them more accessible and more evenly distributed for optical, electrochemical or other measurement. Thus, lysing of blood cells can be a helpful step in creating improved conditions for better analyte measurement.

In many of the embodiments discussed herein, measurements can be taken from a sample of bodily fluid. In some embodiments whole blood can be used. For some analyte measurements, characteristics of the sample can impede measurement. For example, blood can include some analytes in the red blood cells and some analytes in white blood cells. Some analytes can be present in the blood and not present within either type of blood cell. Other, non-analyte components in whole blood may have chemical bonds with similar vibrational frequencies to those of the analyte. Analytes can be unhelpfully shielded (optically, physically, or both) by non-analyte components. Indeed, in some cases analytes can be located within cell membranes and therefore more difficult to measure or quantify, either because they are not evenly distributed, because they are chemically bound, or for some other reason. Other particles within a sample (e.g., blood) can interfere with the measurement, thereby reducing accuracy of the system. For example, in embodiments that use an optical measurement system, red blood cells, or other particles, can absorb, reflect, scatter, or otherwise interfere with the light that is transferred through the sample. Thus, in some embodiments, it can be advantageous to remove these interfering particles or to mitigate or suppress their adverse effects on the measurement. Similarly, it can be advantageous to break down or adjust biological or physiological structures in order to remove optical or physical barriers to measurement, evenly distribute analytes, or otherwise improve analyte detection and measurement. For example, blood can be separated using a centrifuge or filter into components that are organized by similar mass. Blood can also be separated using a lysing process that breaks down the blood's structure on a more fundamental level, breaking cell membranes and causing the contents of cells to be released into a more general suspension. Separation and/or lysing can occur using many mechanical and chemical approaches. For example, cells can be broken down using sonication, heat, lasers, ultrasound, physical shaking (e.g., using a piezoelectric vibrator), homogenization, freeze-thaw procedures, grinding, detergents or other chemical approaches, enzymatic cell disruption, buffers, bacterial or other biological cell lysates, etc. An ultrasound source can focus its energy on a portion of the fluid network to accomplish the lysing function. The ultrasound source can be incorporated into a permanent instrument and aim for a flow cell or other fluid repository contained within a disposable portion configured to interface therewith. Separation into components by mass can occur through settling, centrifugation, etc. Separation by mass can occur before or after cell disruption (e.g., by lysing). The system can include an ultrasound bubble detector. In some cases an ultrasound device can contact the flow cell, and can be used to clean the flow cell. If an ultrasound device is employed for one purpose, it may also be used for one or more purposes. For example, an ultrasound device may help clean fluid passages and may also be used for lysing fluid components prior to measurement.

A fluid handling system (e.g., that shown in FIG. 16) can prepare a sample for measurement. For example, a sample can be prepared by causing interfering substances or particles to move away from an analyte. In some embodiments, the fluid handling system can lyse the red blood cells in a portion of bodily fluid that will be used for the measurement, thereby releasing analytes from being confined within cells. The fluid handling system can include an ultrasound source configured to direct ultrasound energy into the bodily fluid to lyse the red blood cells. The red blood cells can be lysed using any other suitable manner as well, such as the mechanical, sonic, chemical, or optical approaches listed above. Particles other than red blood cells in the sample fluid can also be lysed or broken up using the ultrasound energy or in another manner. In some embodiments, the lysed red blood cells (or other particles) interfere less with the measurement than in their whole state.

Once the red blood cells have been lysed, the cells' cytoplasm and other contents can be released and intermingle with other blood components (e.g., the blood plasma). In some embodiments, the cytoplasm and other lysed components of the red blood cells (or other lysed particles) can interfere with the measurement. Thus, it can be advantageous in some embodiments to remove the red blood cells, or other undesirable particles, from the blood plasma (e.g., using filtering or centrifuging). In some embodiments, the separation of the blood plasma can be performed by the fluid handling system in lieu of lysing the particles as discussed above. In other embodiments, the fluid handling system can be configured to both lyse particles in the sample fluid and also separate the sample into components (e.g., by centrifuging) before or after lysing.

Lysing or another type of separation can provide the advantage of substantially isolating a component of the fluid so that a measurement can be made in a component of the fluid without other components influencing the measurement. If the lysed cells are blood cells, for example, lysing can release cytoplasm and other cell contents from the cell membranes into the blood plasma. Then by centrifugation or filtering, one component of the cells (e.g., the cytoplasm) can be substantially isolated, thereby improving the ability to measure one component (e.g., the cytoplasm) without being interference or obstruction from other components (e.g., cell membranes). Centrifugation can stratify the components into layers. However, centrifugation before lysing can form different layers than centrifugation after lysing. For example, centrifugation after lysing may involve additional substances having their own distinct mass or other physical qualities, resulting in additional strata containing particular cell components of similar mass. In some embodiments, if the cells are not lysed, the accuracy of measurements taken on components inside the cells (e.g., cytoplasm) can be reduced by the cell membranes or other cell components. By lysing the cells, a cell component to be measured (e.g., cytoplasm) can be more easily isolated and measured.

Thus, in some embodiments, a first analysis portion of the fluid sample is prepared for analysis by lysing cells, and a second analysis portion of the fluid sample is prepared for analysis by separating the fluid into a plurality of components (e.g., by centrifugation, filtering, or some other selective process based on mass, size, magnetics, electrical qualities, etc.). In some embodiments, a single analysis portion of the fluid sample can be prepared for analysis by both lysing cells in the fluid and by separating the fluid into a plurality of components. Lysing and/or component separating (e.g., by centrifuging), if warranted, can be performed in series on the same portion of the sample or in parallel on different portions of the sample, or on different samples.

A sample measurement device can include various electrochemical measurement structures described above with respect to FIGS. 1-4. For example, blood can flow through a measurement cell or container that facilitates rapid, minimally perturbed flow with its consistent cross-section and/or smooth interior walls. Such a measurement cell can allow for insertion of membrane technology that is calibrated or otherwise configured to permit flow of molecules through or across a membrane. A measurement strip can be included, and a layer can filter out larger particles while allowing particles of interest to pass through. The particles of interest can be allowed to chemically interact (e.g., using a glucose-oxidase reaction). Electrical leads can be introduced to contact particles of interest or the results of this reaction, and electrical current flowing between these leads can be proportional to amount of the analyte of interest. Thus, the described systems can efficiently bring blood into contact with a measurement device and carry away unused or leftover results after the measurement occurs. The bubble sensors 1816, the loop 1834, the valves 1826, 1832, 1830, 1828, 1854, 1856, 1860, 1858, and the other fluid tubing and control structures shown can all cooperate (e.g., as described above with respect to FIG. 3) to make this possible. Various structures can cooperate to prevent damaged biological fluid from being returned to its source. For example, lysed blood may be harmful if returned to a patient. A waste container 1864 can be especially useful as a repository for cells and biological material that has been lysed, for example.

Use of Systems with EMR and Big Data, Artificial Intelligence

This disclosure refers to the “OptiScanner.” This is a general term used to refer to the analyte systems for analyte measurement featured in this disclosure. A specific example of an OptiScanner is the “OptiScanner 5000 Glucose Monitoring System,” an automated, bedside glucose monitoring device indicated for detecting trends and tracking patterns in persons (age 18 and older) in the surgical intensive care unit. The system collects a venous whole blood sample via connection to a central venous catheter, centrifuges the sample, and measures the plasma glucose concentration. It is not intended for the screening or diagnosis of diabetes mellitus but is indicated for use in determining dysglycemia. The OptiScanner 5000 Glucose Monitoring System is for in vitro diagnostic use. As noted above, the analysis systems (e.g., the OptiScanner) described herein can be especially useful for “detecting trends and tracking patterns.”

U.S. Pat. No. 6,931,328 and U.S. Patent Pub. No. 2007/0083160 are incorporated herein by reference for all purposes, for all that they contain. The former describes an analyte monitoring instrument having network connectivity and the latter describes how data can be incorporated into patient Electronic Medical Records (EMR), for example. The above-described analyte detection and analysis systems can be similarly connected to a network. The described systems collect data frequently and provide beneficial accuracy for the analytes they detect and analyze. For example, some embodiments obtain glucose, lactate, hemoglobin, and/or other data multiple times per hour. This can result in highly valuable data inputs for an artificial intelligence system or other big data analysis server. These data can be collected and analyzed to identify medical or environmental issues that might not be immediately clear to a doctor reviewing a trend of data points for a single variable.

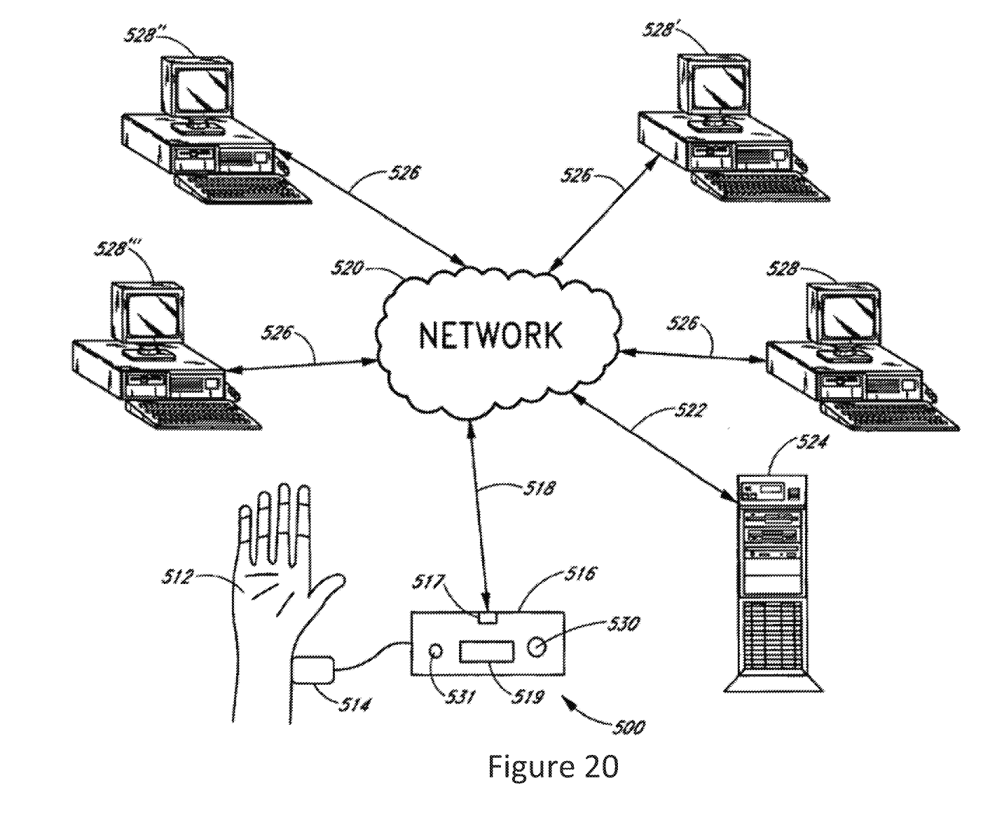

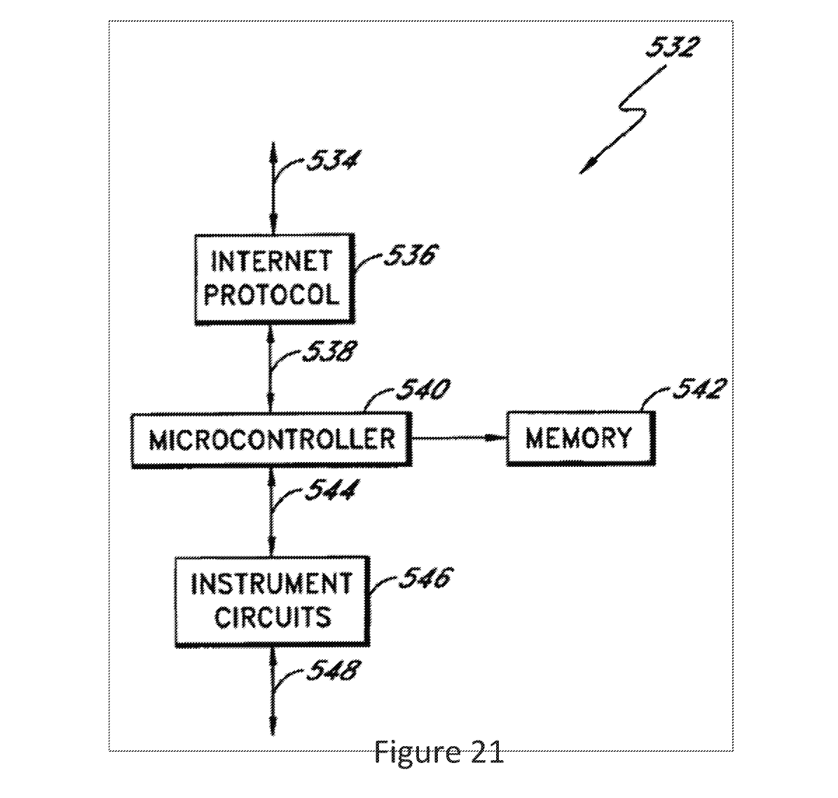

Referring to FIG. 20, an analyte detection system 500 is shown connected to remote stations 524, 528 over a network 520, which may comprise one or more wireless or hardwired links, or a combination of wireless and hardwired links, and/or the Internet. In the illustrated embodiment, the analyte detection system 500 comprises the noninvasive system 10 described above. In other embodiments, the analyte detection system may comprise any other suitable noninvasive system such as (but not limited to) those described in U.S. Pat. No. 5,900,632 to Sterling et al. and U.S. Pat. No. 5,615,972 to Braig et al., the entirety of each of which is hereby incorporated by reference herein and made a part of this specification. In still other embodiments, the analyte detection system 500 may comprise any suitable invasive system such as (but not limited to) the whole-blood system 200 disclosed above.

Because the noninvasive system 10 is depicted in the embodiment of FIG. 20, the hand and forearm 512 of a patient is shown positioned to allow a measurement to be performed by an analyte detector element 514 of the analyte detection system 500. The analyte detection system 500 includes a signal processing system 516 which collects the measurements and/or other suitable information from the detector element 514 and processes the data into a set of results. In the illustrated embodiment, the analyte detection system 500 includes input and output devices, such as a display and a set of control inputs (not shown) for communicating information directly to and from the patient. The signal processing system 516 is equipped with a network interface 517 along with one or more processing elements 519 for processing the measurement signals and for control of network communications.

Data is communicated over the network 520 as determined by the configuration of the system 500 and the state and condition of the measurement being performed. Measurement data may accordingly be communicated to the remote station(s) 524, 528 at the time the measurement is performed, or it may be retained within the system 500 and sent to the remote station(s) according to a schedule or other selection criterion. The system 500 and/or remote station(s) 524, 284 may be capable of comparing each measurement with a set of limits and providing alerts to a supervisory authority regarding excursions therefrom.

In FIG. 20 the measurement data is shown passing through a connection 518 to the network 520, and from there through another connection 522 to a centralized monitoring computer 524 or to a server. The centralized computer 524 may be capable of checking the data for emergency conditions and logging the data for later use. In addition, the centralized computer 524 may monitor the status of the system(s) 500 for proper operation and calibration. It will be appreciated that multiple centralized computers 524 or servers can be provided for communicating with the system(s) 500. In one embodiment, the network interface 517 is a wireless interface (and the connection 518 is a wireless connection), such as but not limited to a Bluetooth interface, an IEEE 802.11(b) interface or a cellular interface, implemented through appropriate hardware built into the system 500.

Furthermore, the centralized computer 524 may simultaneously transfer or route the data (e.g., measurements, system status, etc.) via connection 526 to a computer 528 in the office of a medical practitioner over the network 520. Instead of or in addition to the medical practitioner computer 528, the network may include connections to a computer 528′ located at the manufacturer of the analyte detection system 500, to a computer 528″ located at the patient's home, and/or to a computer 528′″ located at the home or place of business of a parent of the patient. Alternatively, the data may be directly sent over the network 520 to the medical practitioner 528/manufacturer 528′/patient's home 528″/patient's parent 528′″ from the signal processing system 516; in this instance the centralized computer 524 is not necessary and may be omitted from the network 520. Where the centralized computer 524 is omitted, any of the computer(s) 528/528′/528″/528′″ (hereinafter, collectively “528”) may be capable of checking the data received from the system 500 for emergency conditions, logging the data for later use, and/or monitoring the status of the system 500 for proper operation and calibration. It will be appreciated that the foregoing data routing is provided as an example, and not as a limitation, of the data routing utilized to provide the network services in support of a patient's use of the system 500.

In one embodiment, the system 500 includes a panic button 530 which permits the patient to alert a medical practitioner should an important concern arise. In addition, sound and/or visual output may be provided by the system 500 for signaling the patient when the time arrives to perform a measurement, or of a directive from a supervisory authority as received over the network 520.

In another embodiment, the system 500 includes a location button 531 which permits the patient to signal his or her location (as well as the location of the system 500) to any of the remote station(s) 524, 528. When so signaled, a remote user at a remote station 524/528 can direct emergency assistance to the location of the patient/system, should the remote user discover that the patient's condition merits immediate medical attention. In one embodiment, the location information is generated via GPS (Global Positioning System) equipment built into the system 500 and accessible by the processing element(s) 519. In another embodiment, the system 500 continually, intermittently or otherwise automatically transmits its location to any or all of the remote station(s) 524, 528, and the location button 531 may be omitted. In still another embodiment, the system 500 is configured to transmit its location to remote station(s) 524, 528 in response to a query sent from the remote station(s) to the system 500.

In another embodiment, the GPS equipment is supplemented by storage, within appropriate memory accessible by the processing element(s) 519 and/or the GPS equipment, of favorite locations frequented by the patient. Examples of favorite locations include Home, Work, School, etc. and/or a widely recognizable expression thereof, such as the associated street address, nearest cross streets, ZIP or postal code, and/or longitude and latitude. The purpose of such storage is to counteract the tendency of GPS equipment to lose contact with the GPS satellite(s) when the GPS device in question is located inside of a building or other large structure.

Accordingly, when the system 500 loses contact with the GPS satellite(s) and a need arises, under any of the circumstances discussed herein, to transmit the location of the patient/system to a remote user, the system 500 recognizes the loss of contact with the GPS satellite(s) and selects for transmission one of the patient's favorite locations based on the last GPS-computed position of the user/system prior to loss of contact with the GPS satellite(s). In one embodiment, the system 500 selects and transmits whichever favorite location is nearest the last GPS-computed position of the system 500. In another embodiment, the system 500 selects and transmits this nearest favorite location only when the nearest favorite location is within a given minimum distance (e.g., 10 miles, 5 miles, 1 mile, 0.5 miles) from the last GPS-computed position of the system 500. In still another embodiment, the system 500 displays a list of the patient's stored favorite locations on a suitable display, and the patient can select, using an appropriate input device (keypad, button, touchscreen, mouse, voice recognition system, etc.) built into or connected to the system 500, his or her present location from a list of favorites and prompt the system 500 to transmit the selected location.

Any of the location-transmission processes discussed above may be implemented in an algorithm or program instructions executable by, and residing within memory accessible by, the processing element(s) 519 of the system 500 (in particular, by the signal processor 74/260 where the system 500 comprises the noninvasive system 10 or the whole-blood system 200, respectively).

In any of the embodiments discussed herein, the system 500 and/or one or more of the remote station(s) 524, 528 may be configured to encrypt any or all of the data that it transmits over the network 520. Where the user of any of the system 500 and the remote station(s) 524, 528 (or the system/remote station itself) is authorized to receive, read and/or otherwise use the encrypted data, the recipient system 500/remote station 524, 528 is configured to decrypt the encrypted data, to make the data available to the device and/or the user thereof. By encrypting the data, physician-patient confidentiality, or any physician-patient privilege may be preserved, preventing unauthorized reading or use of the data. Encryption also permits transmission of data over wireless networks or public networks such as the Internet while preserving confidentiality of the transmitted data.

It is contemplated that the encryption and decryption may be performed in any suitable manner, with any suitable methods, software and/or hardware presently known or hereafter developed. In the system 500, the encryption and/or decryption processes may be implemented in an algorithm or program instructions executable by, and residing within memory accessible by, the processing element(s) 519 of the system 500 (in particular, by the signal processor 74/260 where the system 500 comprises the noninvasive system 10 or the whole-blood system 200, respectively). In the remote station(s) 524, 528, the encryption and/or decryption processes may be implemented in an algorithm or program instructions executable by, and residing within memory accessible by, processing element(s) (not shown) of the remote station 524/528 in question.

The connection of the system 500 to the network 520, provides either a direct or indirect link from the patient to the practitioner. The practitioner is thereby accorded an ability to monitor the status of the patient and may elect to be alerted should deviations in the measurement values the or timeliness thereof arise. The system may be configured to transmit measurement data at predetermined intervals, or at the time each measurement is performed. The measurements can be transmitted using various network protocols which include standard internet protocols, encrypted protocols, or email protocols.

In one embodiment, the signal processing system 516 is additionally capable of providing visual or audible cues to the patient when the time arrives to conduct a measurement. These alerts may be augmented by requests, transmitted over the network 520 to the instrument, from the practitioner. Errors introduced within measurements and recordation within a manual system can thereby be eliminated with the electronically logged measurements. It will be appreciated that the system provides enhanced utility and measurement credibility in comparison to the use of an instrument that requires manual logging of the measurements and permits no practitioner interaction therewith.

Secretive non-compliance may also be eliminated as the patient is not conferred the responsibility of manually logging measurements. In using the system 500, the measurements collected within the instrument by the patient are capable of being transmitted to a practitioner, or a centralized computer, such that if a patient is not being diligent in conducting measurements, the practitioner may immediately contact the patient to reinforce the need for compliance. In addition, the information provided over the network can be used to warn the practitioner when measurement readings appear abnormal, so that the practitioner may then investigate the situation and verify the status of the patient.

It will be appreciated that the invention has particular utility for patients preferring to receive direct guidance from a practitioner. The information that flows between the patient and the practitioner increases the ability of the practitioner to provide knowledgeable patient guidance.

FIG. 21 illustrates the functional blocks of an embodiment of circuitry 532 for implementing the signal processing hardware 516 shown in FIG. 20. A network connection 534 connects to a network processing circuit, exemplified by an Internet Protocol (IP) circuit or processor 536. Numerous circuits are available for providing internet connectivity, such as the SX-Stack™ chip from Scenix Semiconductor, and the iChip™ from Connect One Electronics. These integrated circuit chips and other available chips provide interface layers for supporting a Transmission Control Protocol/Internet Protocol (TCP/IP). The internet protocol chip 536 has an interface 538 with a control processor section 540, which preferably comprises a microcontroller or the like. The control processor section 540 in turn has access to conventional memory 542. To provide security and fault tolerance of the instrument it is preferable for the control processor, or the internet protocol circuit, to encrypt and provide verification strings or tokens within the data being sent across the network, and accordingly to decrypt information being received and verify the received strings or tokens. The control processor 540 has an interface 544 with the instrumentation circuits 546, which is in turn configured with an interface 548 to the analyte detection element 514 shown in FIG. 20.

The network link provides a mechanism to facilitate performing and recording analyte measurements under supervision, while it additionally provides for periodic instrument calibration, and the ability to assure both measurement and calibration compliance. Calibration data can be communicated from systems 500 in the field to the system manufacturer, or a service organization, so that the systems 500 and their calibrations may be logged. The disclosed network link can be utilized to provide various mechanisms for assuring calibration compliance. Generally the mechanisms are of two categories, those that provide information or a warning about calibration, and those that prevent use of an instrument which is out of calibration. In one embodiment, systems 500 which have exceeded their calibration interval, or schedule, are to be locked out from further use until recalibration is performed. For example, the system 500 may be set to operate for thirteen months for a given calibration interval of twelve months. The system 500 may issue warnings prior to the expiration of calibration, and warnings of increased severity after the expiration of the calibration interval. If the system 500, however, is not properly calibrated by the end of the thirteen months, normal operation ceases, thereby locking out the user after providing an appropriate error message in regard to the expired calibration. Upon recalibration, the calibrated operation interval is restored to provide for another thirteen month period of calibrated operation.

Alternatively, or in addition thereto, a “lockout command” can be sent to the system 500 over the network 520 from the manufacturer, practitioner or system maintenance organization, thereby engaging a lockout mode of the system 500, so that operation may not be continued until the system 500 has been serviced. The lockout command could also be sent in the event that the patient has not paid his or her bills, or be sent under other circumstances warranting lockout of the system 500.

Another mode is that of locking out normal system use after the expiration of calibration, and allowing limited use thereafter only after a code, or token, has been downloaded from a supervisory site. Although many variations are possible, the code could for instance be provided when a calibration appointment is made for the system 500. To provide continued service and minimize cost, the patient may be allowed to perform calibration checks of the system 500. The patient is supplied with a small set of analyte calibration standards which are read by the system 500 once it is put into a calibration mode and preferably connected to a remote site for supervising the process. Should the calibration check pass, wherein the instrument readings fall within normal levels, or be capable of being automatically adjusted thereto, the calibration interval may be extended. Failure of the calibration check would typically necessitate returning the system 500 for service.

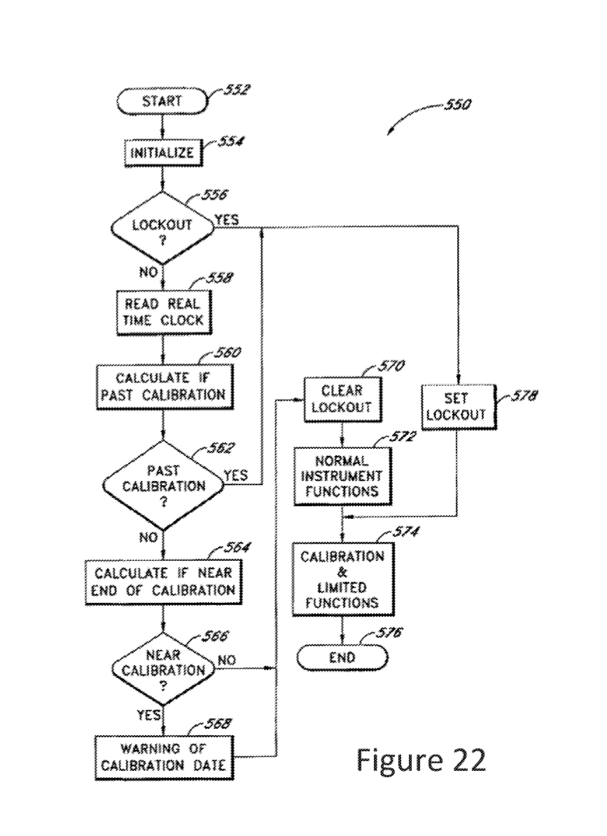

FIG. 22 illustrates an embodiment of a process 550 for assuring calibration compliance within the analyte detection system 500 by utilizing a lockout mechanism. The programmed instructions associated with the analyte detection system 500 are started at block 552 and initialized at block 554, and a check is made on a lockout flag at block 556 to determine if it was set during a prior session by a command received via the network 520, or due to being out of calibration. Not having been locked out from a prior session, the real-time clock (RTC) of the system 500 is read at block 558 and a calculation is performed at block 560 comparing the current date with the stored calibration date and calibration interval. If upon checking calibration at block 562 the calibration interval has not yet expired, then a calculation is performed at block 564 comparing the current date with the stored calibration date and near-calibration interval. Near-calibration is checked at block 566 and, if calibration is to expire soon, then a user warning is issued at block 568, preferably informing the user of the date of the upcoming expiration of the calibration interval. The lockout flag is cleared at block 570 and processing within the system 500 continues with normal instrument functions being accessible at block 572, along with calibration and other limited functions at block 574, until the user shuts down the instrument and processing ends at block 576. If the lockout flag was set from a prior instrument operation, or the calibration interval was exceeded, then a lockout flag would be set at block 578, and the instrument functionality would thereby be restricted to execution of the calibration procedures and other limited functions at block 578 while the normal instrument functionality would not be accessible. The calibration procedure itself may be augmented and improved by providing interaction between the servicing party and the manufacturer, such interaction may include providing guidance information to the servicing party, and the collection of measurement information by the manufacturer.

It will be appreciated that the present invention provides functionality beyond that which can be provided by a stand-alone analyte detection system, as the practitioner, or practitioner's office, is involved in the analyte measurement process to confer a portion of the benefits normally associated with an office visit. The aforesaid description illustrates how these features provide the capability for two-way data flow which facilitates the conducting and recording of correct measurements while encouraging compliance in regard to both measurements and instrument calibration. Furthermore, the data collected by the system may be utilized by others in addition to the practitioner, such as pharmaceutical companies which may be provided data access to alter or administer medication programs, and insurance companies which may require data regarding patient diligence according to the specified treatment program.

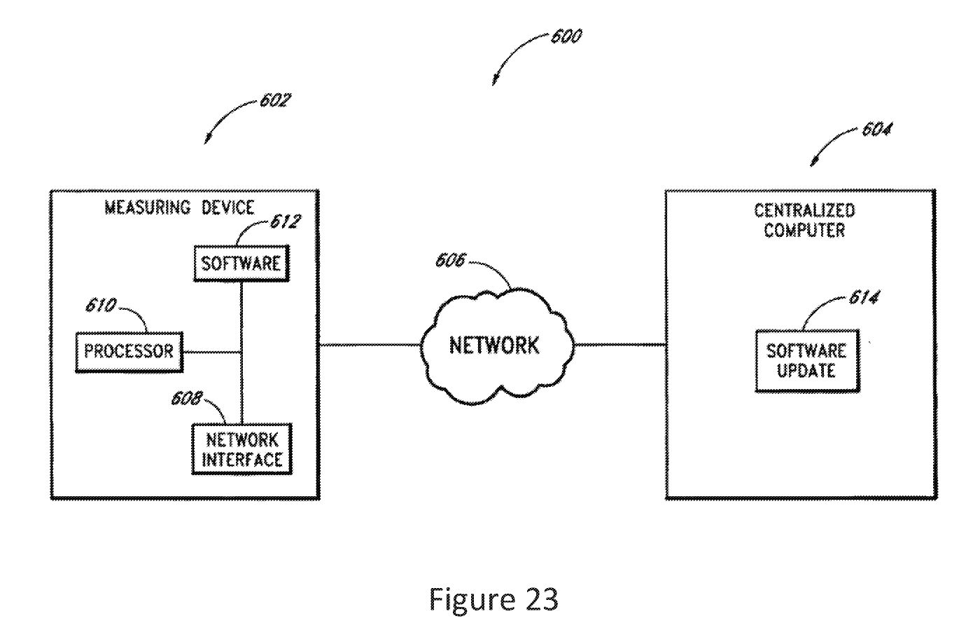

FIG. 23 illustrates one embodiment of a software update system 600. The software update system 600 includes an analyte detection system 602 that is connectable to a centralized computer 604 via a network 606. The analyte detection system 602 may comprise a portable, near-patient device that is capable of optically measuring analytes in a material sample. Other examples of the analyte measuring device 602 include, but not limited to, the noninvasive system 10 discussed in this disclosure, the whole-blood system 200 discussed in this disclosure, or any other suitable invasive or noninvasive analyte detection system.

As used herein, the term “computer” is a broad term and is used in its ordinary sense and refers, without limitation, to any programmable electronic device that can store, retrieve and process data. Examples of computers include terminal devices, such as personal computers, workstations, servers, mini computers, main-frame computers, laptop computers, a network of individual computers, mobile computers, palm top computers, hand held computers, set top for a TV, an interactive television, an interactive kiosk, a personal digital assistant (“PDA”), an interactive wireless communications device, or a combination thereof. The computers may further possess storage devices, input devices such as a keyboard, mouse or scanner, and output devices such as a computer screen or a speaker. Furthermore, the computers may serve as clients, servers, or a combination thereof.

As used herein, the term “network” is a broad term and is used in its ordinary sense and refers, without limitation, to a series of points or nodes interconnected by communication paths, such as a group of interconnected computers. Examples of networks are the Internet, storage networks, local area networks and wide area networks.

Further to FIG. 23, the analyte detection system 602 includes a network interface 608, a processor 610 and software 612. The centralized computer 604 includes at least one software update 614. In one embodiment, when the analyte measuring device 602 is connected to the network 606, the analyte detection system 602 and the centralized computer 604 are in two-way communication. Consequently, the analyte detection system 602 may send information (e.g., analyte measurements) to the centralized computer 604 and the centralized computer 604 may send information (e.g., a software update 614) to the analyte measuring device 602. Other architectures of networked systems, as known by those skilled in the art, may also be used in place of the architecture set forth in FIG. 23. For example, the architecture shown in FIG. 22, and/or any of the variants thereof discussed herein. For example, one or more of the computers 528/528′/528″/528′″ may share the herein-described functions of, or replace entirely, the centralized computer 604, in which case the centralized computer 604 may be omitted.

As used herein, the term “processor” is a broad term and is used in its ordinary sense and refers, without limitation, to the part of a computer that operates on data. Examples of processors are central processing units (“CPU”) and microprocessors.

As used herein, the term “software” is a broad term and is used in its ordinary sense and refers, without limitation, to instructions executable by a computer or related device. Examples of software include computer programs and operating systems.

As used herein, the term “software update” or “update” is a broad term and is used in its ordinary sense and refers, without limitation, to information used by a computer to modify software. A software update may be, for example, data, algorithms or programs.

A process flow diagram of a preferred software update process 700 is shown in FIG. 24. First, in an act 702, a user performs analyte measurements with the analyte detection system 602. Advantageously, as discussed above, the user may perform analyte measurements using the analyte detection system 602 at a remote location (e.g., the user's home).

Further to the act 702, the analyte detection system 602 detects analytes in a material sample and calculates an analyte concentration in accordance to the analyte detection system's software 612. Additionally, the analyte detection system may issue alerts to the user, for example, in response to exceeded tolerances defined in the software 612. The alerts may be visually displayed to the user and/or audibly sounded to the user. For instance, the analyte detection system 602 may issue an alert in response to an elapsed calibration time tolerance defined in the software 612. Other alerts may be issued when the software or analyte-concentration calculation algorithm is out of date, or when the analyte concentration reading made by the detection system 602 are higher or lower than defined safe limits or ranges.

In one embodiment, the software 612 is contained in the analyte detection system 602 internally. In another embodiment, the software 612 is retained external to the analyte detection system 602.

Next, in an act 704, the analyte detection system 602 is connected to the centralized computer 604 via the network 606. Advantageously, the network interface 608 readily connects the analyte detection system 602 to the network 606. Furthermore, once the analyte measuring device 602 is connected to the network 606, the analyte measuring device 602 is, in one embodiment, in two-way communication with the centralized computer 604. In one embodiment, the communication between the analyte measuring device 602 and the centralized computer 604 is established without any intervention from a user.

The process 700 then proceeds to a decision act 706 where the centralized computer 604 determines an update status for the analyte measuring device's software 612. Various conditions may trigger the centralized computer 604 to update the software 612. In one embodiment, a condition for updating the software 612 is the presence of a new drug in the material sample (e.g., a new drug taken by the user) that alters the analyte calculations. Specifically, the centralized computer 604 determines whether the software 612 currently in use accounts for the use of the new drug. If the current software does not account for the new drug, the centralized computer 604 sends a software update 614 over the network 706 that does account for the new drug, and as a result, corrects future analyte calculations performed by the analyte measuring device 602. In another embodiment, a condition for updating the software 612 is where a new analyte-detection algorithm is developed. For example, the new algorithm may improve the accuracy or speed of the analyte detection system 602 over the software 612 currently in use. In another embodiment, a condition for updating the software 612 is where the analyte detection system 602 should display a new warning or where the monitoring device should display an existing warning in response to new or different events. The existing warning or the new warning may be displayed, for instance, in response to new information learned from a subset of a customer population. Advantageously, other conditions not specifically mentioned herein may also trigger the centralized computer 604 to update the software 612.

If the centralized computer 604 decides that the software 612 does not need to be updated in the decision act 706, then the update process 700 proceeds via the “No” path to an act 708. In the act 708, the user disconnects the analyte detection system 602 from the network and the software 612 is not updated. Thus, the analyte detection system 602 operates in the same manner as the analyte detection system 602 previously operated in the act 702.

If the centralized computer 604 decides that the software 612 needs to be updated in the decision act 706, then the update process 700 proceeds via the “Yes” path to an act 710. In the act 710, the centralized computer 604 sends a software update 614 to the analyte detection system 602. In one embodiment, the centralized computer 604 contains a database of various software updates 614, and consequently, the centralized computer 604 selects the appropriate software update 614 from the database and then sends the software update 614 to the analyte detection system 602.

Next, in an act 712, the analyte detection system 602 receives (e.g. downloads) the software update 614. The analyte detection system 602 then preferably modifies the software 612 to an updated version of the software 612. The process then proceeds to an act 714.

In the act 714, the user performs analyte measurements in accordance with the updated software 612. Thus, depending upon the software update 614, the analyte measuring device 602 operates differently than the manner in which the analyte measuring device 602 previously operated in act 702. One example is that the analyte detection system 602 may calculate analyte concentrations differently. Another example is that the analyte detection system 602 may displays new warnings to the user. A further example is that the analyte detection system 602 may display the same warnings, but the warnings are triggered by different events.

In any of the embodiments of the software update system 600 discussed herein, the analyte detection system 602 and/or the centralized computer 604 (or, where applicable, the computer(s) 528) may be configured to encrypt any or all of the data that it transmits over the network 606. Where the user of any of the analyte detection system 602 and the centralized computer 604 (or the analyte detection system/centralized computer itself) is authorized to receive, read and/or otherwise use the encrypted data, the recipient system 602/computer 604 is configured to decrypt the encrypted data, to make the data available to the device and/or the user thereof. By encrypting the data, physician-patient confidentiality, or any physician-patient privilege may be preserved, preventing unauthorized reading or use of the data. Encryption also permits transmission of data over wireless networks or public networks such as the Internet while preserving confidentiality of the transmitted data.

It is contemplated that the encryption and decryption may be performed in any suitable manner, with any suitable methods, software and/or hardware presently known or hereafter developed. In the analyte detection system 602, the encryption and/or decryption processes may be implemented in an algorithm or program instructions executable by, and residing within the memory accessible by, the processor 610 of the analyte detection system 602 (in particular, by the signal processor 74/260 where the analyte detection system 602 comprises the noninvasive system 10 or the whole-blood system 200, respectively). In the centralized computer 604 (or, where applicable, the computer(s) 528), the encryption and/or decryption processes may be implemented in an algorithm or program instructions executable by, and residing within memory accessible by, processing element(s) (not shown) of the computer 604/528 in question.

The software update process 700 has many advantages. One advantage is that the software 612 of the analyte measuring device 602 may be updated without requiring significant user participation. Another advantage is that the software 612 may be quickly and conveniently updated at a remote location (e.g., the user's home) rather than requiring the user to travel to, for example, a doctor's office or other administrative center.

There are many challenges and risks involved with the naïve use of Big Data in medical settings without accompanying knowledge of medicine, focusing on issues in critical care. For example, there is a challenge of transforming the large amount of data into usable and useful medical knowledge. The system described here helps address these challenges in several ways. First, it provides very useful, frequent, accurate inputs to a big data analysis. Second, it can provide a window into this specific, timely information for doctors directly in the critical care environment. The described analyte detection system itself does not suffer from the risks of big data discussed in the article because its outputs are specific and depend on analysis and algorithms that are well understood. By integrating this described system into a Big Data system, it can help anchor big data algorithmic or predictive outputs in reliable hard data. By comparing, contrasting, or juxtaposing data from a single analyte measurement system (e.g., an “OptiScanner,” which can refer to a device having features disclosed herein) connected to a single patient, for example, with output from a large number of analyte measurement systems (e.g., OptiScanners), system administrators and doctors can identify medical and/or technical issues.

The principles discussed above can be used, for example, in Intensive Care Unit (ICU) scoring systems such as APACHE (Acute Physiology and Chronic Health Evaluation), MPM (Mortality Probability Model), and SAPS (Simplified Acute Physiology Score). These systems have use as short-hand indicators of patient acuity. Because the described analyte measuring systems improve data accuracy (and increase data volume and resolution greatly by using periodic measurements), these systems can greatly improve these scoring system values. Another database that can be improved with the described analyte monitor is the Multiparameter Intelligent Monitoring in Intensive Care (MIMIC) database and the like. For example, the described analyte measurements systems can provide“dynamic clinical data mining” (DCDM). Thus, they can assist in a process where users of EMR can be automatically presented with prior interventions and outcomes of similar patients to support medical decisions.

Another benefit of providing network connectivity and access to big data algorithms and outputs is that the described systems provide an interface to doctors with the knowledge to weigh risks and who are present to act quickly on big data outputs, in view of analyte system outputs. For example, in some embodiments, a portion of an analyte measurement system (e.g., OptiScanner or similar analysis instrument having features disclosed herein and using enzymatic technology, sometimes referred to as an “OptiZymer” device) can provide visual outputs to a medical professional through a graphic user interface, or GUI. Example GUIs are described, for example, in U.S. Publication No. 2015/0045641 and in U.S. Publication No. 2008/0077073. The entire contents of these publications are incorporated herein for all purposes, for all that they contain. Some embodiments have the benefit of receiving user input, where a doctor can annotate a visual display to indicate and record where or when a patient received a dose of medication, when a medical event occurred that may affect the data, a prediction based on an emerging trend, a warning to disregard an outlier data point, etc. A portion of the GUI (or an alternative or additional GUI view that is readily accessible therefrom) can be provided to show output or results from a big data algorithm or to display results from a different source outside of the main instrument. Thus, a doctor can be alerted to an issue or a trend among other similarly-situated patients, or an issue or trend with similar instruments. Making such additional information available readily, in close association with hard data specific to the patient, can allow a doctor to identify relevant actions to be taken, thereby alleviating some of the problems with pure big data applications that do not involve medical knowledge or do not involve the context of accurate data readings.

As more robust big data outputs are developed, the described GUI and system can be incorporated as an alert system not only for outputs from the device itself, but also for outputs from trusted big data sources. For example, if medical professionals determine as a group that a specific lactate level or trend should be investigated immediately based on common patient morbidity after those levels or trends are analyte measurement system (e.g., OptiScanner) GUI can incorporate this big data output into a visual warning sign for a doctor in the hospital room of such a patient. This warning can be based on both the actual readings from the local analysis instrument, as well as the latest trend or level warning data available from big data sources, which can communicate such big data information over an internet connection to an analyte measurement system (e.g., OptiScanner or OptiZymer, for example). The GUI can rapidly indicate the source of a warning to medical professionals. For example, one portion of the interface can be designated for display of data sourced purely from the instrument itself, and another portion can be designated for display of outputs from external algorithms (which may include inputs from the device). To reduce screen size, these portions can be alternatively accessible based on user input. To avoid mistakes, the GUI can have a starkly different appearance based on the source of the information displayed (different colors, fonts, arrangement of information, numerical values, etc.)

An example of how big data outputs can be enhanced using the systems described herein is described with respect to FIGS. 25-27. FIG. 25 is a flowchart that schematically illustrates an embodiment of an analysis method 2800. Embodiments of the method 2800 can be used to diagnose the presence and/or severity of a disease state in a patient. In other embodiments, the method 2800 may be used to identify one or more disease state indicators in a repository of biological fluid (e.g., a blood sample stored in a test tube in one implementation). In the embodiment illustrated in FIG. 25, in block 2810 a collection of infrared spectroscopic data may be obtained for a sample population of ICU patients having one or more specific, diagnosed disease states. A collection of spectra for a population of normal patients (e.g., without an indication of the presence of any of the disease states) may also be obtained. Based on patient diagnoses, the sample population of spectra can be classified into subpopulations of spectra from patients having one or more disease states. For example, in one illustrative sample population, patient diagnoses were available for the following disease states shown in the following table:

Disease State

Sepsis - with or without any complicating factors
Sepsis - without any organ failure
Sepsis - without indication of hetastarch
Liver failure - without indication of kidney failure
Kidney failure - without indication of liver failure
Liver and Kidney failure

As can be seen from the table, in this illustrative example the subpopulation having sepsis (with or without any complicating factors) was broken into two further subpopulations: those without organ failure and those without indication of hetastarch (a plasma expander commonly administered to patients after trauma, blood loss, or serious injury). The disease states shown in the table are intended to be illustrative and not to limit the scope of the disclosed systems and methods for diagnosing disease states. In other examples, a population of sample data may be classified into additional and/or different disease states for analysis. For example, a population of patients having the sepsis disease state may be broken into subpopulations corresponding to bacterial infection, fungal infection, or both types of infection. Other disease states and sample populations may be used with other embodiments of the disclosed systems and methods.

In one example of the method 2800, collections of spectra (for both the ICU population and the normal population) were obtained from patient blood plasma samples using a Fourier Transform Infrared (FTIR) spectrometer sensitive to the mid-infrared spectral region between about 7 microns and about 10 microns. In other examples, a different spectral region may be used (e.g., near-infrared, far-infrared, visible, etc.). Other types of spectral data (e.g., narrow-band and/or broad-band spectra) and/or spectroscopic instruments may be used in other embodiments. For example, in some implementations, spectral data taken at one or more discrete wavelength passbands may be used (see, e.g., the description of the spectroscopic analyzer 2010 shown in FIG. 19).

In the embodiment of the method 2800 shown in FIG. 25, in block 2820 the collection of spectra can be processed by removing the contribution of glucose from individual spectra. For example, a reference glucose spectrum can be derived from a spectrum of an aqueous glucose solution measured on the same spectroscopic instrument that measured the population spectra. A glucose-free spectrum may be obtained by subtracting the reference glucose spectrum multiplied by a scaling coefficient. The glucose-free spectra of the sample population of ICU patients will be denoted by So, and the glucose-free spectra of the normal population of patients will be denoted by N₀. In block 2830, a difference spectrum Do is calculated as the difference between the glucose-free sample spectra and the glucose-free normal spectra: D₀=S₀−N₀.

Continuing with the embodiment of the method 2800, in block 2840, the difference spectra are analyzed to determine a set of basis spectra that span the space of the difference spectra Do. For example, in some embodiments, a non-parametric method such as principle component analysis (PCA) can be used to determine the basis spectra. In some such embodiments, a singular value decomposition (SVD) of the matrix of difference spectra is performed (typically after subtraction of the mean) according to:

D₀=A B C^{T},

where A and C are orthogonal matrices and B is a diagonal matrix. The columns of the matrix A are called the principal components (PC) of the difference spectra Do, and they form a set of orthonormal basis spectra that can be used to parameterize the space of differences between the (mean-centered) glucose-free sample spectra So and the (mean-centered) glucose-free normal spectra N₀. In some embodiments, a commercially-available mathematical analysis package such as, e.g., MATLAB®, is used for the PCA computations.

In other embodiments of the method 2800, the basis spectra can be determined (in block 2840) by other statistical techniques including, but not limited to, factor analysis, kernel methods (e.g., kernel PCA), independent component analysis, and/or other techniques from pattern analysis.

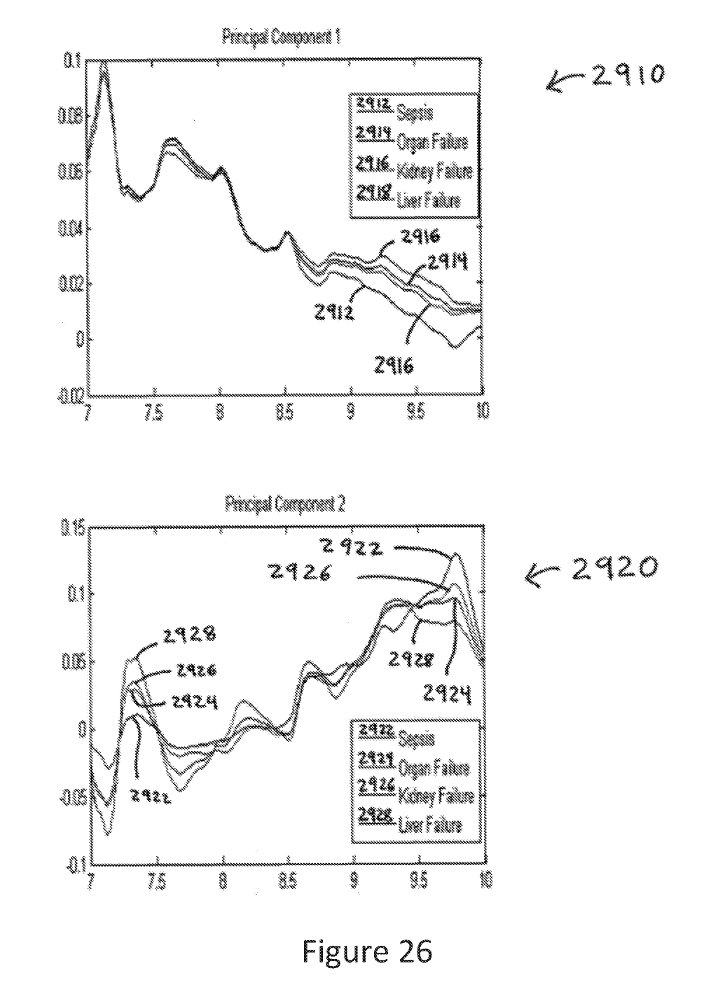

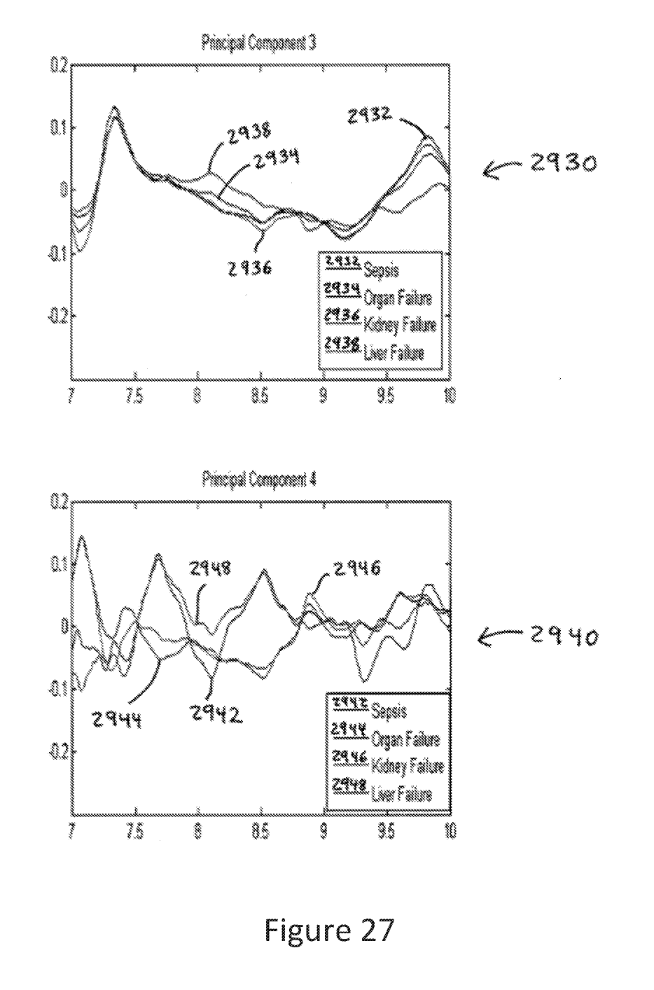

FIGS. 26-27 illustrate example graphs 2910-2940 showing the first four principle components of difference spectra obtained according to an embodiment of the method 2800. The horizontal axes of these graphs are wavelength in microns. The graphs 2910, 2920, 2930, and 2940 in FIGS. 26-27 show the first, second, third, and fourth principle components, respectively. The different curves in each graph correspond to the principal component for populations having different disease states, namely, sepsis, organ failure (both liver and kidney failure), kidney failure (without liver failure), and liver failure (without kidney failure). In this illustrative example, significant similarity was found among the principal components for the populations with sepsis (with and without complicating factors), sepsis (without organ failure), and sepsis (without hetastarch). Therefore, only one sepsis curve is shown in each of the graphs of FIGS. 29A and 29B. The following table lists reference numbers identifying the disease states for the curves shown in the graphs 2910-2940.

| Disease State | Reference numerals in FIGS. 28-29 |
|---|---|
| Sepsis | 2912, 2922, 2932, 2942 |
| Organ Failure | 2914, 2924, 2934, 2944 |
| KidneyFailure | 2916, 2926, 2936, 2946 |
| Liver Failure | 2918, 2928, 2938, 2948 |

Without subscribing to or requiring any particular theory or interpretation, the example graphs 2910-2940 illustrate some of the following features, which may or may not be present (or present to the same degree) in examples based on different sample populations or for different disease states. In this example, the first principal components 2912-2918 for the populations with the different disease states are relatively similar in shape and magnitude across the wavelength range shown in FIG. 26. Additionally, the first principle component for a mean-centered population of normal spectra was calculated and was found to be similar in shape and magnitude to the first principle components 2912-2918 shown in FIG. 26. A possible explanation for this similarity is the predominance of proteins in the spectral makeup of blood plasma (whether from normal or sick individuals) as well as the relatively larger magnitude of variation in protein concentrations, compared to all other blood components. Thus, it is possible that the general similarity of the first principal components may reflect the variation of protein concentrations present in the normal population, since plasma samples from patients with disease states will also include this “normal” variation.

In certain embodiments, this “normal” variation seen in the first principle component (e.g., possibly due to normal blood protein variation) may be reduced by using signal processing techniques that reduce the variation in the difference spectra due to this “unwanted” component. For example, in certain such embodiments, the normal variation is treated as a “noise” component that is removed by signal processing techniques such as, e.g., filtering, Fourier analysis, etc. By accounting for and removing some or all of the normal variation found in plasma spectra, such embodiments may better extract the underlying signal corresponding to disease-state specific features of the principal components. Further, such embodiments may provide increased contrast between features in one or more principal components that correspond to different disease states (e.g., the contrast between a sepsis-specific feature and a kidney-failure-specific feature).

FIG. 27 shows that the second principle component 2922 for sepsis is substantially distinct from the second principle components 2924-2928 for organ failure, kidney failure, and liver failure, which are relatively similar to each other. The second principle component 2922 for sepsis was also found to be highly correlated with a kinked-feature near 8 microns found in certain patient spectra. The kinked feature includes a positive portion from about 8.5 microns to about 10 microns that is highly correlated with the spectrum of hetastarch, and a negative portion from about 7 microns to about 8.5 microns that resembles a main protein peak with smaller constituents. In some patient spectra, the presence of the kinked feature in the spectrum may be related to administration of hetastarch with fluid replacement in order to staunch blood loss from the patient. In such patients, the blood loss may lead to a negative relative concentration of blood proteins (compared to a normal population) and the administration of hetastarch may lead to a positive relative concentration of hetastarch (compared to a normal population). Additionally or alternatively, it is possible that the presence of the kinked feature in patient spectra may be related to the second principal component for sepsis and may provide a diagnostic indication of the presence (and/or severity) of sepsis in the patient.

FIG. 27 shows that the third and the fourth principle components 2932-2938 and 2942-2948 begin to diverge from each other and show recognizable differences among the populations with the sepsis, organ failure, kidney failure, and liver failure populations. Higher-order principle components (e.g., fifth, sixth, seventh, etc., which are not shown in FIGS. 29A and 29B) tend to show a zig-zag appearance as a function of wavelength. Certain embodiments of the disclosed systems and methods may use information in the principle components (including higher-order principle components up to, e.g., the sixth or the seventh) to reduce glucose estimation error.

Returning to the embodiment of the method 2800 shown in FIG. 28, in block 2840, the basis spectra (e.g., PCs) are determined that span the space of the difference spectra. The number N_{B} of basis spectra generally is equal to the number of intensity values of the spectra in the Sample and Normal Populations. For example, if embodiments of the spectroscopic analyzer 2010 (FIG. 18) are used, the number N_{B} may be equal to the number of finite-bandwidth infrared filters used (e.g., 25 in one implementation). In embodiments in which an FTIR spectroscope is used, the number N_{B} may be larger, as such instruments often provide very high-resolution spectra.

In some embodiments, the basis spectra are rank-ordered from 1 to N_{B}. For example, it is common in PCA to rank order the principle components in descending order of their associated variances (obtainable from the diagonal matrix B). For example, the principle components shown in FIGS. 29A and 29B have been rank-ordered by variance. As discussed above, in some embodiments, a subset N_{D} of the basis spectra may provide useful diagnostic information related to disease states. For example, in some embodiments, the subset includes the first 4 principal components (N_{D}=4). In other embodiments, a different number of principal components may be used such as, for example, 1, 2, 3, 5, 6, 7, 8, 9, or more. In some embodiments, the number N_{D} can be selected by choosing the principle components having associated variances that are larger than a threshold value. In other embodiments, the subset can include all N_{B} of the principle components (N_{D}=N_{B}) or can include all the principle components up to the rank r of the covariance matrix (N_{D}=r).

In block 2850 of the method 2800 shown in FIG. 25, a patient spectrum is provided for diagnosis of possible disease states. The patient spectrum may be measured by the same instrument that measured the collection of sample and normal population spectra or it may be measured by a different instrument. In some embodiments, the patient spectrum may be included in the sample (or normal) population of spectra. The patient spectrum is compared with the subset of basis spectra to provide diagnostic information for the presence (and/or severity) of one or more disease states. If the number of data points in the patient spectrum is different from the number of data points in the basis spectra, interpolation, curve-fitting, splines, or other suitable procedures may be used to provide the comparison. In some embodiments, other data or signal processing techniques may be used including, for example, filtering, averaging, smoothing, etc.

In certain embodiments, the patient spectrum is initially processed to provide a glucose-free, mean-subtracted patient spectrum for comparison with the difference spectra (determined in block 2830). The correction for glucose in the patient spectrum may be determined using the same or similar techniques as for the glucose-free spectra in the Sample and Normal populations. For example, a scaled, reference glucose spectrum may be subtracted from the patient spectrum to provide a glucose-free patient spectrum. In other embodiments, the glucose-free patient spectrum may be determined using embodiments of the methods for analyte determination described above. For example, in some implementations, an embodiment of the method 2100 described above with reference to FIG. 21 is used to determine for the patient spectrum an estimate for glucose concentration (with or without correction for possible interferents). The estimated glucose concentration can be used to provide the glucose-free patient spectrum (e.g., by subtracting a reference glucose spectrum scaled by the estimated glucose concentration).

In block 2860, the likelihood of the presence (and/or severity) of one or more disease states is determined using one or more statistical tests based at least in part on a comparison of the patient spectrum and the subset that includes N_{D} of the basis spectra. As described above, in some embodiments, a glucose-free, mean-subtracted patient spectrum may be used for the comparison.

In some embodiments, the statistical test includes convolving a probability density function (PDF) of a statistical distribution function of the “distance” between the patient spectrum and a linear combination of the subset of basis vector with a PDF for the basis spectra. For example, the distance may be measured according to the Mahalanobis distance (or distance squared) or some other suitable statistical distance metric (e.g., Hotelling's T-square statistic). In some such embodiments, the statistical test includes calculating the following integral over the subspace spanned by the subset N_{D} of the basis spectra (represented by the N_{D}-dimensional variable v):

∫PDF[MD²(X−A□v)]PDF[v]dv.

In this convolution integral, MD² represents the square of the Mahalanobis distance. The argument of MD² is the difference between the patient spectrum X and the inner (dot) product between the first N_{D} columns of the orthogonal matrix A (obtained from the SVD of the difference spectra) and the vector v . The PDF of MD² is multiplied by the PDF of the basis spectra PDF[v] and integrated over the subspace spanned by the subset of basis spectra. In some embodiments, PDF[v] is an N_{D} dimensional Gaussian PDF having mean and variance obtainable from the PCA analysis (e.g., the variances for the basis spectra are found from the diagonal matrix B).

In some embodiments of the method, the convolution integral is evaluated using the basis spectra corresponding to a specific disease state (e.g., sepsis). The value of the convolution integral can be used to determine the likelihood of the presence and/or severity of the disease state. For example, in some implementations, a binary statistical test is performed such that if the value of the convolution integral exceeds a threshold, then the patient is diagnosed with the disease state. If the value of the convolution integral is below the threshold, the patient is not diagnosed with the disease state. In some such implementations, an amount by which the convolution value exceeds the threshold is used to provide an indication of the severity of the disease state in the patient. In some embodiments, the convolution integral is evaluated for each of several disease states (e.g., sepsis, organ failure, etc.). As discussed, a binary statistical test may be used to diagnose the presence (and/or severity) of each of the disease states. In some implementations, the values for the convolution integrals for each of the disease states are rank ordered, and a relative likelihood of the presence (and/or severity) of the disease states is provided. For example, some such implementations may provide information that it is more likely that sepsis is present than is kidney failure and so forth.

In some embodiments of the method, other statistical tests may be used. For example, Baye's law may be used to interpret the results of the convolution analysis. In some embodiments, Baye's law may provide an indication of the likelihood of the presence (and/or severity) of a disease state, given the prior information corresponding to the patient spectrum. In other embodiments, the results of the statistical analysis may be combined with information on concentrations of one or more analytes. For example, results of the statistical analysis for the kidney failure disease state may be combined with information on concentration of creatinine (and/or urea) in a patient fluid sample to provide an improved diagnosis of the presence (and/or severity) of kidney failure and/or renal dysfunction.

In some embodiments, the apparatus 100 shown in FIG. 1 advantageously may be used to withdraw a fluid sample (e.g., a blood sample) from the patient for analysis by the monitoring device 102. The monitoring device 102 may be configured to provide the patient spectrum of the fluid sample (or of a component of the sample such as, e.g., plasma) and perform the statistical analysis for disease states. For example, the fluid handling system 404 (FIG. 4) may be used to withdraw a bodily fluid sample from the patient and transport the sample to the optical system 412 for determination of the patient spectrum by spectroscopic analysis. The algorithm processor 416 can receive the patient spectrum and perform the statistical tests for the presence and/or severity of one or more disease states. In some embodiments, the algorithm processor 416 stores some or all of the information used for the statistical tests including, for example, the subset of basis spectra, the probability density functions, the variances of the basis spectra, etc. The algorithm processor 416 may include software code modules configured to evaluate the convolution integral above using, for example, suitable numerical methods. In some embodiments, the software code modules (and/or data used by the code modules) are stored on a computer-readable medium such as, e.g., volatile or non-volatile memory, random access memory, optical media such as a CD-ROM or a DVD, semiconductor memory, etc.

In some embodiments, the display system 414 (FIG. 4) may be used to display information related to the results of the analysis for disease states. For example, the user interface 2400 (FIG. 24) may be configured to display an indication of the presence and/or severity of one or more disease states. The user interface 2400 may display a relative likelihood of one or more disease states. In some cases, information relating to the diagnosed disease states is stored in a storage medium, communicated over a network to another processor and/or storage medium, and/or used in further diagnosis methods. In some cases, information relating to the diagnosed disease states is communicated over a hospital information system and stored in a database comprising patient information. Information relating to the diagnosed disease states may be used by health care providers to update, change, and/or initiate a treatment procedure for the disease state.

In some implementations, if the disease state is determined to be sufficiently severe, an alert may be communicated to appropriate health care providers. For example, the alert may comprise an audible and/or visible signal. The alert may comprise an alert message communicated to an attending physician via a suitable network (e.g., the hospital information system). Some embodiments of the apparatus 100 may be configured to take other actions in response to the diagnosis of a disease state. For example, the apparatus 100 may initiate (or change the rate of) infusion of an appropriate infusion fluid or medicament to the patient.

In other embodiments, other processors or computer systems (e.g., the computer system 2646) may be configured to perform some or all of the analysis for disease states. In some such embodiments, the monitoring apparatus 104 is configured to take the patient spectrum (e.g., an infrared spectrum) and communicate information related to the patient spectrum to other processors for analysis. In some such embodiments, the other processors may store the information related to the subset of basis spectra and so forth. Many variations are possible and the above examples of possible system implementations are intended to be illustrative.

This describes how spectra or other data can be obtained from sample populations with and without a disease state. Such disease states can include sepsis, organ failure, kidney failure, and liver failure, for example. The data can be evaluated algorithmically (e.g., a glucose-free spectrum can be obtained, and a difference calculated between the spectra or other data for the two populations). Statistical methods such as component analysis (using linear algebra to develop basis sets, etc.) can be used to determine a likelihood of the presence of a disease state. This type of statistical analysis can be an input into a big data network. The systems described herein can both deliver and receive input for (or results from) analyses such as this. Another example of how the system can provide data for important medical analysis, diagnosis, and treatment is provided herein. This describes how data taken from the described systems, and potentially related systems, can be combined to determine important parameters such as cardiac output.

In some embodiments, an apparatus such as those described herein can assist in warning a physician (or patient, nurse, or other user) regarding one or more danger signs of a harmful medical condition. An example of such a condition is sepsis. Because many of the described embodiments are configured to be located close to (and indeed connected to) a patient in an intensive care setting and have access to a patient's vital biological information, it can be a useful tool for such warnings. In addition to measuring glucose and lactate levels, for example, such a device can also measure core body temperature (e.g., using a temperature sensor in a patient connector that is exposed to or otherwise measures patient blood soon after it exits a patient's central vein, for example, as described elsewhere herein). In the cases of sepsis, data regarding respiration rate, heart rate, and core temperature can be received by (or generated by sensors or other apparatus within) a device. Sepsis can be predicted and/or imminent when respiration and hear rates both increase, in connection with changes in core temperature of a patient. Thus, when data between devices and/or portions of a device are shared and such a medical event is present or recorded, a doctor may be warned, e.g., using a GUI such as those referred to above.

### EXAMPLES

In 1st Example, an analyte detection system comprises: a fluid passageway having a patient end, the fluid passageway configured to provide fluid communication with a bodily fluid in a patient via the patient end; a pump in fluid communication with the fluid passageway, the pump configured to withdraw bodily fluid from the patient via the patient end of the fluid passageway; and a flow cell in fluid communication with the fluid passageway and the pump, the flow cell configured to receive the bodily fluid withdrawn from the patient, the flow cell comprising: a first opening proximal to the patient end and in fluidic communication with the fluid passageway; a second opening distal to the patient end and in fluidic communication with the pump; a flow cell chamber in communication with the first opening and the second opening, the flow cell chamber comprising: an analysis region configured to detect presence of at least one analyte in the withdrawn bodily fluid, wherein the analysis region comprises at least one enzymatic sensor configured to detect presence of the at least one analyte; and an optical investigation region transmissive to visible light, the optical investigation region configured to be in optical communication with an optical system comprising a visible light source and an optical detector.

In a 2nd example, the analyte detection system of Example 1, wherein the at least one enzymatic sensor comprises a glucose sensor or a lactate sensor.

In a 3rd example, the analyte detection system of any of Examples 1-2, wherein the at least one analyte comprises glucose or lactate.

In a 4th example, the analyte detection system of any of Examples 1-3, wherein the withdrawn bodily fluid comprises whole blood.

In a 5th example, the analyte detection system of Example 4, wherein the optical investigation region is configured to measure a level of Hemoglobin in the whole blood.

In a 6th example, the analyte detection system of any of Examples 1-5, wherein the optical system is configured to inclusion in the flow of the withdrawn bodily fluid.

In a 7th example, the analyte detection system of any of Examples 1-6, wherein the analysis region comprises at least one membrane configured to allow diffusion of the at least one analyte from the withdrawn bodily fluid towards the enzymatic sensor.

In a 8th example, the analyte detection system of Example 7, wherein the at least one membrane is configured to allow diffusion of the at least one analyte from the withdrawn bodily fluid towards the enzymatic sensor such that an electrical output of the enzymatic sensor reaches a saturation level in a time interval less than or equal to about 1 minute.

In a 9th example, the analyte detection system of any of Examples 1-8, wherein the cross-sectional area of the flow cell chamber is approximately equal to the cross-sectional area of the first or the second opening.

In a 10th example, the analyte detection system of any of Examples 1-9, further comprising a length of tubing greater than five feet disposed between the second opening of the flow cell and the pump, the length of tubing configured to prevent flow of the withdrawn bodily fluid into the pump.

In a 11th example, the analyte detection system of any of Examples 1-10, wherein the withdrawn fluid is returned to the patient after analysis.

In a 12th example, a method of measuring concentration of at least one analyte in a bodily fluid comprises: withdrawing a sample of bodily fluid from a patient via a patient end of a fluid passageway; drawing the sample of withdrawn bodily fluid into a flow cell, the flow cell comprising a region transmissive to visible light; measuring the concentration of at least one analyte in the sample of withdrawn bodily fluid using an enzymatic sensor provided within the flow cell; measuring the concentration of hemoglobin in the sample of withdrawn bodily fluid using an optical system configured to transmit visible light through the region of the flow cell transmissive to visible light; and returning the sample of withdrawn bodily fluid to the patient.

In a 13th example, the method of Example 12, wherein the sample of bodily fluid is withdrawn using a pump.

In a 14th example, the method of any of Examples 12-13, wherein the concentration of the at least one analyte in the sample of withdrawn bodily fluid using an enzymatic sensor is measured in a time interval less than or equal to 1 minute.

In a 15th example, the method of any of Examples 12-14, wherein the optical system comprises a photodetector configured to detect light scattered or transmitted through the sample of withdrawn bodily fluid.

In a 16th example, a flow cell configured to analyze bodily fluid and determine concentration of at least one analyte in the bodily fluid comprises: an inlet configured to allow flow of a sample of the bodily fluid therethrough; an outlet configured to allow flow of the sample of the bodily fluid therethrough; a flow cell chamber in fluid communication with the inlet and the outlet, the flow cell chamber comprising: an analysis region configured to detect presence of at least one analyte in the withdrawn bodily fluid, wherein the analysis region comprises at least one enzymatic sensor configured to detect presence of the at least one analyte; and an optical investigation region transmissive to visible light, the optical investigation region configured to be in optical communication with an optical system comprising a visible light source and an optical detector.

In a 17th example, the flow cell of Example 16, comprising a moldable material.

In a 18th example, the flow cell of any of Examples 16-17, wherein the analysis region comprises at least one membrane configured to allow diffusion of the at least one analyte from the withdrawn bodily fluid towards the enzymatic sensor.

In a 19th example, the flow cell of any of Examples 16-18, wherein the at least one membrane is configured to allow fast diffusion of the at least one analyte from the withdrawn bodily fluid towards the enzymatic sensor such that an electrical output of the enzymatic sensor reaches a saturation level in a time interval less than or equal to about 1 minute.

In a 20th example, the flow cell of any of Examples 16-19, wherein the enzymatic sensor comprises at least one of a glucose sensor or a lactate sensor.

In a 21th example, the flow cell of any of Examples 16-20, wherein at least one enzymatic sensor configured to detect presence of the at least one analyte comprises both an enzymatic lactate sensor and an enzymatic glucose sensor.

In a 22nd example, the flow cell of any of Examples 16-21, wherein a cross-sectional area of the flow chamber is substantially equal to a cross-sectional area of the first inlet/outlet or the second inlet/outlet.

In a 23rd example, a method of manufacturing a flow cell comprises: providing a first section comprising: a first semi-cylindrical opening at a first end of the first section and a second semi-cylindrical opening at a second end of the first section; and a widened region connected to the first and the second semi-cylindrical openings, the widened region comprising a slot for receiving an enzymatic sensor, at least a portion of the widened region comprising a material transmissive to visible light; providing a second section comprising: a first semi-cylindrical opening at a first end of the second section and a second semi-cylindrical opening at a second end of the second section; and a widened region connected to the first and the second semi-cylindrical openings, the widened region comprising a slot for receiving an enzymatic sensor, at least a portion of the widened region comprising a material transmissive to visible light; and attaching the first section and the second section such that: the first semi-cylindrical openings of the first and the section sections form a first tubular opening, the second semi-cylindrical openings of the first and the section sections form a second tubular opening, and the widened regions of the first and the section sections form a widened chamber, wherein a cross-sectional area of the widened chamber is substantially equal to the cross-sectional areas of the first and the second tubular openings.

In a 24th example, the method of manufacturing the flow cell of Example 23, wherein attaching the first section and the second section comprises laser welding the first and the second sections.

In a 25th example, the method of manufacturing the flow cell of any of Examples 23-24, wherein a dimension of the widened region is less than 250 microns.

### TERMINOLOGY AND CONCLUSION

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

Embodiments of the disclosed systems and methods may be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules, but may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

A number of applications, publications, and external documents may be incorporated by reference herein. Any conflict or contradiction between a statement in the body text of this specification and a statement in any of the incorporated documents is to be resolved in favor of the statement in the body text.

Although described in the illustrative context of certain preferred embodiments and examples, it will be understood by those skilled in the art that the disclosure extends beyond the specifically described embodiments to other alternative embodiments and/or uses and obvious modifications and equivalents. Thus, it is intended that the scope of the claims which follow should not be limited by the particular embodiments described above.

What is claimed is:

1. An analyte detection system comprising:
a fluid passageway having a patient end, the fluid passageway configured to provide fluid communication with a bodily fluid in a patient via the patient end;
a pump in fluid communication with the fluid passageway, the pump configured to withdraw bodily fluid from the patient via the patient end of the fluid passageway; and
a flow cell in fluid communication with the fluid passageway and the pump, the flow cell configured to receive the bodily fluid withdrawn from the patient, the flow cell comprising:
a first opening proximal to the patient end and in fluidic communication with the fluid passageway;
a second opening distal to the patient end and in fluidic communication with the pump;
a flow cell chamber in communication with the first opening and the second opening, the flow cell chamber comprising:
an analysis region configured to detect presence of at least one analyte in the withdrawn bodily fluid, wherein the analysis region comprises at least one enzymatic sensor configured to detect presence of the at least one analyte; and
an optical investigation region transmissive to visible light, the optical investigation region configured to be in optical communication with an optical system comprising a visible light source and an optical detector.

2. The analyte detection system of claim 1, wherein the at least one enzymatic sensor comprises a glucose sensor or a lactate sensor.

3. The analyte detection system of claim 1, wherein the at least one analyte comprises glucose or lactate.

4. The analyte detection system of claim 1, wherein the withdrawn bodily fluid comprises whole blood.

5. The analyte detection system of claim 4, wherein the optical investigation region is configured to measure a level of Hemoglobin in the whole blood.

6. The analyte detection system of claim 1, wherein the optical system is configured to inclusion in the flow of the withdrawn bodily fluid.

7. The analyte detection system of claim 1, wherein the analysis region comprises at least one membrane configured to allow diffusion of the at least one analyte from the withdrawn bodily fluid towards the enzymatic sensor.

8. The analyte detection system of claim 7, wherein the at least one membrane is configured to allow diffusion of the at least one analyte from the withdrawn bodily fluid towards the enzymatic sensor such that an electrical output of the enzymatic sensor reaches a saturation level in a time interval less than or equal to about 1 minute.

9. The analyte detection system of claim 1, wherein the cross-sectional area of the flow cell chamber is approximately equal to the cross-sectional area of the first or the second opening.

10. The analyte detection system of claim 1, further comprising a length of tubing greater than five feet disposed between the second opening of the flow cell and the pump, the length of tubing configured to prevent flow of the withdrawn bodily fluid into the pump.

11. The analyte detection system of claim 1, wherein the withdrawn fluid is returned to the patient after analysis.

12. A method of measuring concentration of at least one analyte in a bodily fluid, the method comprising:
withdrawing a sample of bodily fluid from a patient via a patient end of a fluid passageway;
drawing the sample of withdrawn bodily fluid into a flow cell, the flow cell comprising a region transmissive to visible light;
measuring the concentration of at least one analyte in the sample of withdrawn bodily fluid using an enzymatic sensor provided within the flow cell;
measuring the concentration of hemoglobin in the sample of withdrawn bodily fluid using an optical system configured to transmit visible light through the region of the flow cell transmissive to visible light; and
returning the sample of withdrawn bodily fluid to the patient.

13. The method of claim 12, wherein the sample of bodily fluid is withdrawn using a pump.

14. The method of claim 12, wherein the concentration of the at least one analyte in the sample of withdrawn bodily fluid using an enzymatic sensor is measured in a time interval less than or equal to 1 minute.

15. The method of claim 12, wherein the optical system comprises a photodetector configured to detect light scattered or transmitted through the sample of withdrawn bodily fluid.

16. A flow cell configured to analyze bodily fluid and determine concentration of at least one analyte in the bodily fluid, the flow cell comprising:
an inlet configured to allow flow of a sample of the bodily fluid therethrough;
an outlet configured to allow flow of the sample of the bodily fluid therethrough;
a flow cell chamber in fluid communication with the inlet and the outlet, the flow cell chamber comprising:
an analysis region configured to detect presence of at least one analyte in the withdrawn bodily fluid, wherein the analysis region comprises at least one enzymatic sensor configured to detect presence of the at least one analyte; and
an optical investigation region transmissive to visible light, the optical investigation region configured to be in optical communication with an optical system comprising a visible light source and an optical detector.

17. The flow cell of claim 16, comprising a moldable material.

18. The flow cell of claim 16, wherein the analysis region comprises at least one membrane configured to allow diffusion of the at least one analyte from the withdrawn bodily fluid towards the enzymatic sensor.

19. The flow cell of claim 16, wherein the at least one membrane is configured to allow fast diffusion of the at least one analyte from the withdrawn bodily fluid towards the enzymatic sensor such that an electrical output of the enzymatic sensor reaches a saturation level in a time interval less than or equal to about 1 minute.

20. The flow cell of claim 16, wherein the enzymatic sensor comprises at least one of a glucose sensor or a lactate sensor.

21. The flow cell of claim 16, wherein at least one enzymatic sensor configured to detect presence of the at least one analyte comprises both an enzymatic lactate sensor and an enzymatic glucose sensor.

22. The flow cell of claim 16, wherein a cross-sectional area of the flow chamber is substantially equal to a cross-sectional area of the first inlet/outlet or the second inlet/outlet.

23. A method of manufacturing a flow cell, the method comprising:
providing a first section comprising:
a first semi-cylindrical opening at a first end of the first section and a second semi-cylindrical opening at a second end of the first section; and
a widened region connected to the first and the second semi-cylindrical openings, the widened region comprising a slot for receiving an enzymatic sensor, at least a portion of the widened region comprising a material transmissive to visible light;
providing a second section comprising:
a first semi-cylindrical opening at a first end of the second section and a second semi-cylindrical opening at a second end of the second section; and
a widened region connected to the first and the second semi-cylindrical openings, the widened region comprising a slot for receiving an enzymatic sensor, at least a portion of the widened region comprising a material transmissive to visible light; and
attaching the first section and the second section such that:
the first semi-cylindrical openings of the first and the section sections form a first tubular opening,
the second semi-cylindrical openings of the first and the section sections form a second tubular opening, and
the widened regions of the first and the section sections form a widened chamber, wherein a cross-sectional area of the widened chamber is substantially equal to the cross-sectional areas of the first and the second tubular openings.

24. The method of manufacturing the flow cell of claim 23, wherein attaching the first section and the second section comprises laser welding the first and the second sections.

25. The method of manufacturing the flow cell of claim 23, wherein a dimension of the widened region is less than 250 microns.
